(19) United States
(12) Patent Application Publication
(10) Pub. No.: US 2026/0069596 A1
(43) Pub. Date: Mar. 12, 2026

(54) USE OF PLK1 INHIBITOR AS MONOTHERAPY AND IN COMBINATION WITH CETUXIMAB IN TREATING RAS WILD-TYPE COLORECTAL CANCER

(71) Applicant: Cardiff Oncology, Inc., San Diego, CA (US)

(72) Inventors: Maya Ridinger, San Diego, CA (US); Tod Smeal, San Diego, CA (US); Mark Erlander, San Diego, CA (US)

(21) Appl. No.: 19/391,715

(22) Filed: Nov. 17, 2025

Related U.S. Application Data
(63) Continuation of application No. PCT/US2025/018218, filed on Mar. 3, 2025.
(60) Provisional application No. 63/561,215, filed on Mar. 4, 2024.
(60) Provisional application No. 63/575,195, filed on Apr. 5, 2024.

(51) Int. Cl. A61K 31/519; A61K 39/00; A61K 45/06; A61P 35/00; C07K 16/28

(52) U.S. Cl. CPC A61K 31/519; A61K 45/06; A61P 35/00; C07K 16/2863; A61K 2039/505

(57) ABSTRACT

Provided include methods, compositions and kits for treating cancer (e.g., RAS^{WT} colorectal cancer) in a subject. The method can comprise administrating an a PLK1 inhibitor (for example, onvansertib) or a combination of a PLK1 inhibitor and an EGFR inhibitor to the subject in a manner sufficient to inhibit progression of the cancer.

### RELATED APPLICATIONS

This application claims the benefit under 35 U.S.C. § 119 (e) of U.S. Provisional Patent Application Ser. No. 63/561,215, filed Mar. 4, 2024, and U.S. Provisional Patent Application Ser. No. 63/575,195, filed Apr. 5, 2024, the contents of these related applications are incorporated herein by reference in their entirety for all purposes.

### BACKGROUND

### Field

The present disclosure relates generally to the field of treating cancer, more specifically, for treatment of colorectal cancer.

### Description of the Related Art

Previous therapies for metastatic colorectal cancer (mCRC) include cytotoxic chemotherapy combined with targeted therapy against the epidermal growth factor receptor (EGFR, cetuximab and panitumumab) or the vascular endothelial growth factor (VEGF, bevacizumab). EGFR inhibitors (EGFRi) have shown to provide clinical benefit to mCRC patients with RAS wild-type (RAS^{WT}) tumors. However, their clinical benefit is limited due to intrinsic resistance or development of resistance. New therapeutic strategies are needed to prolong the clinical benefit of EGFRi and overcome resistance.

### SUMMARY

Disclosed herein include methods of treating RAS wild type (RAS^{WT}) colorectal cancer. In some embodiments, the method comprises: administering a polo-like kinase 1 (PLK1) inhibitor to a subject with a RAS^{WT} colorectal cancer, thereby inhibiting or reducing progression of the RAS^{WT} colorectal cancer in the subject.

In some embodiments, the subject with the RAS^{WT} colorectal cancer is resistant to or does not respond effectively to an epidermal growth factor receptor (EGFR) inhibitor. In some embodiments, the subject with the RAS^{WT} colorectal cancer is resistant to treatment with the EGFR inhibitor or has stable or progressive disease following treatment with the EGFR inhibitor. In some embodiments, the subject with RAS^{WT} colorectal cancer is resistant to cetuximab, panitumumab, or both; or the subject with the RAS^{WT} colorectal cancer has stable or progressive disease following treatment with cetuximab, panitumumab, or both. In some embodiments, the resistance is acquired resistance or intrinsic resistance.

In some embodiments, the subject with the RAS^{WT} colorectal cancer has received a prior EGFR inhibitor therapy. In some embodiments, the subject with the RAS^{WT} colorectal cancer did not respond to the treatment with the prior EGFR inhibitor therapy. In some embodiments, the subject with the RAS^{WT} colorectal cancer has stable or progressive disease following the treatment with the prior EGFR inhibitor therapy. In some embodiments, the subject with the RAS^{WT} colorectal cancer is known to be resistant to the EGFR inhibitor therapy.

In some embodiments, the PLK1 inhibitor is administered to the subject in a cycle of 7 days, 14 days, 28 days, 35 days, 42 days, or 49 days. In some embodiments, the PLK1 inhibitor is administered to the subject daily. In some embodiments, each cycle of treatment is at least about 14 days. In some embodiments, each cycle of treatment is from about 14 days to about 28 days. In some embodiments, the PLK1 inhibitor is administered on at least five days, at least ten days, or at least fifteen days in a cycle. In some embodiments, the PLK1 inhibitor is not administered on at least one day, at least three days, or at least seven days in a cycle. In some embodiments, the subject undergoes at least two cycles of the administration of the PLK1 inhibitor.

The EGFR inhibitor can be, e.g., erlotinib, lapatinib, AZD8931, WZ4002, panitumumab, vandetanib, icotinib, afatinib, brigatinib, CO-1688, AZD-4769, poziotinib, CUDC-101, S-222611, AC-480, imgatuzumab, sapitinib, TAS-2913, theiiatinib, XGFR-2421, HM-61713B, epitinib, NRC-2694, MLBS-42, JRP-890, cetuximab, AL-6802, TAK-285, BGB-102, AEE788, gefitinib, DMS-3008, TX-2036, KI-6783, KI-6896; or a pharmaceutically acceptable salt, hydrate, solvate or prodrug thereof; or any combination thereof. In some embodiments, the EGFR inhibitor is cetuximab or panitumumab. In some embodiments, the PLK1 inhibitor is onvansertib (NMS-P937), BI2536, volasertib (BI 6727), GSK461364, adavosertib (AZD1775), CYC140, HMN-176, HMN-214, rigosertib (ON-01910), MLN0905, TKM-080301, TAK-960, GTPL10072, Ro3280; or a pharmaceutically acceptable salt, hydrate, solvate or prodrug thereof; or any combination thereof. In some embodiments, the PLK1 inhibitor is onvansertib.

Disclosed herein include methods of treating RAS wild type (RAS^{WT}) colorectal cancer. In some embodiments, the method comprises: administering a PLK1 inhibitor and a first EGFR inhibitor to a subject with the RAS^{WT} colorectal cancer, thereby inhibiting or reducing progression of the RAS^{WT} colorectal cancer in the subject.

In some embodiments, the subject with the RAS^{WT} colorectal cancer is resistant to or does not respond effectively to a second EGFR inhibitor. In some embodiments, the subject with the RAS^{WT} colorectal cancer is resistant to treatment with the second EGFR inhibitor or has stable or progressive disease following treatment with the second EGFR inhibitor. In some embodiments, the subject with RAS^{WT} colorectal cancer is resistant to cetuximab, panitumumab, or both or has stable or progressive disease following treatment with cetuximab, panitumumab, or both. In some embodiments, the resistance is acquired resistance or intrinsic resistance.

In some embodiments, the subject with the RAS^{WT} colorectal cancer has received a prior EGFR inhibitor therapy. In some embodiments, the subject with the RAS^{WT} colorectal cancer has received a prior EGFR inhibitor therapy, wherein the EGFR inhibitor is the second EGFR inhibitor. In some embodiments, the subject with the RAS^{WT} colorectal cancer did not respond to the treatment with the prior EGFR inhibitor therapy. In some embodiments, the subject with the RAS^{WT} colorectal cancer has stable or progressive disease following the treatment with the prior EGFR inhibitor therapy. In some embodiments, the subject with the RAS^{WT} colorectal cancer is known to be resistant to the second EGFR inhibitor.

The PLK1 inhibitor and the first EGFR inhibitor can be co-administered simultaneously. The PLK1 inhibitor and the first EGFR inhibitor can be administered sequentially. In some embodiments, the PLK1 inhibitor, the first EGFR inhibitor, or both are administered to the subject in a cycle of 7 days, 14 days, 28 days, 35 days, 42 days, or 49 days. In some embodiments, the PLK1 inhibitor is administered daily, and the first EGFR inhibitor is administered twice a week. In some embodiments, each cycle of treatment is at least about 14 days. In some embodiments, each cycle of treatment is from about 14 days to about 28 days. In some embodiments, the PLK1 inhibitor is administered on at least five days, at least ten days, or at least fifteen days in a cycle. In some embodiments, the PLK1 inhibitor is not administered on at least one day, at least three days, or at least seven days in a cycle. In some embodiments, the first EGFR inhibitor is administered once or twice weekly. In some embodiments, the first EGFR inhibitor is administered at least once weekly for two, three, four, five, six or seven consecutive weeks in a cycle. In some embodiments, the subject undergoes at least two cycles of the administration of the PLK1 inhibitor and the first EGFR inhibitor.

The first and/or second EGFR inhibitor can be, e.g., erlotinib, lapatinib, AZD8931, WZ4002, panitumumab, vandetanib, icotinib, afatinib, brigatinib, CO-1688, AZD-4769, poziotinib, CUDC-101, S-222611, AC-480, imgatuzumab, sapitinib, TAS-2913, theiiatinib, XGFR-2421, HM-61713B, epitinib, NRC-2694, MLBS-42, JRP-890, cetuximab, AL-6802, TAK-285, BGB-102, AEE788, gefitinib, DMS-3008, TX-2036, KI-6783, KI-6896; or a pharmaceutically acceptable salt, hydrate, solvate or prodrug thereof; or any combination thereof. In some embodiments, the first and/or second EGFR inhibitor is cetuximab or panitumumab. The first and second EGFR inhibitors can the same or different.

The PLK1 inhibitor can be, e.g., onvansertib (NMS-P937), BI2536, volasertib (BI 6727), GSK461364, adavosertib (AZD1775), CYC140, HMN-176, HMN-214, rigosertib (ON-01910), MLN0905, TKM-080301, TAK-960, GTPL10072, Ro3280; or a pharmaceutically acceptable salt, hydrate, solvate or prodrug thereof; or any combination thereof. In some embodiments, the PLK1 inhibitor is onvansertib.

In some embodiments, the subject has received at least one prior cancer treatment. In some embodiments, the prior treatment does not comprise the use of a PLK1 inhibitor. In some embodiments, the PLK1 inhibitor is onvansertib. In some embodiments, the subject was in remission for cancer. In some embodiments, the subject in remission for cancer was in complete remission (CR) or in partial remission (PR). The method can comprise determining cancer status of the subject. The method can comprise determining responsiveness of the subject to the treatment with the PLK1 inhibitor, or treatment with the PLK1 inhibitor and the first EGFR inhibitor.

The method can comprise administering one or more additional cancer therapeutics or therapies for the cancer. In some embodiments, the method does not comprise administration of additional cancer therapeutics or therapies for the cancer. In some embodiments, the additional therapeutics or therapies for cancer comprise chemotherapy, radiation therapy, immunotherapy, hormone therapy, hyperthermia, photodynamic therapy, stem cell therapy, surgery, targeted therapy, or any combination thereof.

The subject can be human. In some embodiments, the subject achieves a complete response. In some embodiments, the volume of one or more tumors in the subject increases by at most 25% relative to the volume of the one or more tumors prior to the administering, following at least one cycle of treatment. In some embodiments, the volume of one or more tumors in the subject decreases by at least 25% relative to the volume of the one or more tumors prior to the administering, following at least one cycle of treatment.

Disclosed herein include methods of sensitizing cancer cells to an EGFR inhibitor. In some embodiments, the method comprises: contacting cancer cells with a composition comprising onvansertib, thereby sensitizing the cancer cells to the EGFR inhibitor.

In some embodiments, the cancer cells are RAS^{WT} colorectal cancer cells. In some embodiments, the EGFR inhibitor is cetuximab or panitumumab. In some embodiments, contacting cancer cells with the composition occurs in vitro, ex vivo, and/or in vivo. In some embodiments, contacting cancer cells with the composition is in a subject. In some embodiments, the subject did not respond to, or is known to be resistant to, the EGFR inhibitor. In some embodiments, the subject had prior treatment with the EGFR inhibitor. In some embodiments, the subject is a mammal. In some embodiments, the mammal is a human.

The method can comprise determining sensitization of the cancer cells to the EGFR inhibitor after being contacted with the composition. The method can comprise contacting the cancer cells with the EGFR inhibitor. In some embodiments, contacting the cancer cells with the EGFR inhibitor occurs in the subject. The method can comprise determining the response of the subject to the EGFR inhibitor. In some embodiments, the volume of one or more tumors comprising the cancer cells in the subject increases by at most 25% relative to the volume of the one or more tumors prior to the contacting. In some embodiments, the volume of one or more tumors comprising the cancer cells in the subject decreases by at least 25% relative to the volume of the one or more tumors prior to the contacting. In some embodiments, contacting the cancer cells with the EGFR inhibitor is concurrent with the contacting the cancer cells with the composition, or after the contacting the cancer cells with the composition. In some embodiments, the cancer cells are cells of EGFR-amplified cancer.

Disclosed herein include kits. The kit can comprise a PLK1 inhibitor. In some embodiments, the kit comprises: a first EGFR inhibitor. The kit can comprise a manual providing instructions for administering the PLK1 inhibitor or co-administering the PLK1 inhibitor and the first EGFR inhibitor to a subject in need thereof for treating RAS^{WT} colorectal cancer.

In some embodiments, the instructions comprise instructions for co-administrating the PLK1 inhibitor and the first EGFR inhibitor simultaneously. In some embodiments, the instructions comprise instructions for co-administrating the PLK1 inhibitor and the first EGFR inhibitor sequentially. In some embodiments, the instructions comprise instructions for administering to a subject that did not respond to treatment with a second EGFR inhibitor alone. In some embodiments, the instructions comprise instructions for administering to a subject resistant to the second EGFR inhibitor. In some embodiments, the first and/or second EGFR inhibitor is erlotinib, lapatinib, AZD8931, WZ4002, panitumumab, vandetanib, icotinib, afatinib, brigatinib, CO-1688, AZD-4769, poziotinib, CUDC-101, S-222611, AC-480, imgatuzumab, sapitinib, TAS-2913, theiiatinib, XGFR-2421, HM-61713B, epitinib, NRC-2694, MLBS-42, JRP-890, cetuximab, AL-6802, TAK-285, BGB-102, AEE788, gefitinib, DMS-3008, TX-2036, KI-6783, KI-6896; or a pharmaceutically acceptable salt, hydrate, solvate or prodrug thereof; or any combination thereof. In some embodiments, the PLK1 inhibitor is onvansertib (NMS-P937), BI2536, volasertib (BI 6727), GSK461364, adavosertib (AZD1775), CYC140, HMN-176, HMN-214, rigosertib (ON-01910), MLN0905, TKM-080301, TAK-960, GTPL10072, Ro3280; or a pharmaceutically acceptable salt, hydrate, solvate or prodrug thereof; or any combination thereof. In some embodiments, the first and/or second EGFR inhibitor is cetuximab or panitumumab and/or the PLK1 inhibitor is onvansertib. In some embodiments, the first and second EGFR inhibitors are the same. In some embodiments, the first and second EGFR inhibitors are different.

### BRIEF DESCRIPTION OF THE DRAWINGS

FIG. 1 displays a exemplary cartoon of generation of RAS^{WT} colorectal patient-derived xenograft (PDX) cancer models and determination of cetuximab sensitivity.

FIG. 2 displays a non-limiting exemplary cartoon of generation of cetuximab acquired resistance.

FIG. 3-FIG. 5 display non-limiting exemplary data related to antitumor activity of onvansertib and cetuximab in RAS^{WT} CRC PDX models sensitive to cetuximab (FIG. 3), or with intrinsic (FIG. 4) or acquired (FIG. 5) resistance to cetuximab. PDX models were treated with vehicle, onvansertib (Onv), cetuximab (Cetux) or the combination (Onv+Cetux) for 18-19 days. Tumor volumes (TV) were measured twice a week, and % tumor volume change (TVC) from baseline was calculated as follows: (TVi/TV0−1)×100. Tumor stasis defined as TVC between 0 and 20%, and tumor regression as TVC less than 0% at last measurement. Results are presented as mean±SEM. Unpaired t-test was used to compare % TVC at last measurement between combination treatment and the most effective monotherapy; *p<0.05, **p<0.01, ***p<0.001, ****p<0.0001.

FIG. 6A-FIG. 6C display non-limiting exemplary data related to antitumor activity of onvansertib monotherapy and in combination with cetuximab across all models. FIG. 6A shows % tumor volume change (TVC) at last measurement for onvansertib group across all models (mean±SEM). FIG. 6B-FIG. 6C show % TVC for onvansertib (Onv), cetuximab (Cetux) and combination (Onv+Cetux) groups for cetuximab sensitive (FIG. 6B) and resistant (FIG. 6C) models. One-way ANOVA with Tukey's multiple comparisons test was used to compare % TVC, *p<0.05, **p<0.01, ****p<0.0001.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein includes methods for treating cancer. The cancer can be colorectal cancer. In some embodiments, the cancer is RAS wild type (RAS^{WT}) colorectal cancer.

Disclosed herein include methods of treating RAS wild type (RAS^{WT}) colorectal cancer. In some embodiments, the method comprises: administering a polo-like kinase 1 (PLK1) inhibitor to a subject with a RAS^{WT} colorectal cancer, thereby inhibiting or reducing progression of the RAS^{WT} colorectal cancer in the subject.

Disclosed herein include methods of treating RAS wild type (RAS^{WT}) colorectal cancer. In some embodiments, the method comprises: administering a PLK1 inhibitor and a first EGFR inhibitor to a subject with the RAS^{WT} colorectal cancer, thereby inhibiting or reducing progression of the RAS^{WT} colorectal cancer in the subject.

Disclosed herein include methods of sensitizing cancer cells to an EGFR inhibitor. In some embodiments, the method comprises: contacting cancer cells with a composition comprising onvansertib, thereby sensitizing the cancer cells to the EGFR inhibitor.

Disclosed herein include kits. In some embodiments, the kit comprises: a PLK1 inhibitor. In some embodiments, the kit comprises: a first EGFR inhibitor. The kit can comprise a manual providing instructions for administering the PLK1 inhibitor or co-administering the PLK1 inhibitor and the first EGFR inhibitor to a subject in need thereof for treating RAS^{WT} colorectal cancer.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e., an oncologist) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal.

As used herein, “administration” or “administering” refers to a method of giving a dosage of a pharmaceutically active ingredient to a vertebrate.

As used herein, a “dosage” refers to the combined amount of the active ingredients (e.g., cetuximab and onvansertib).

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate and the likes. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, each of the terms “partial response,” “partial remission” and “PR” refers to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone marrow transplantation, and immunotherapy. The size of a tumor can be detected by clinical or by radiological means. Tumor-indicating markers can be detected by means well known to those of skill, e.g., ELISA or other antibody-based tests. A partial response of the target lesion can refer to at least a 30% decrease in the sum of the diameters of target lesions, taking as reference the baseline sum diameters.

As used herein, each of the terms “complete response” or “complete remission” or “CR” means that a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence of a tumor can be detected by clinical or by radiological means. Tumor-indicating markers can be detected by means well known to those of skill, e.g., ELISA or other antibody-based tests. A “complete response” does not necessarily indicate that the cancer has been cured, however, a complete response may be followed by a relapse. A complete response of a target lesion includes disappearance of all target lesions and any pathological lymph nodes (whether target or non-target) having reduction in short axis to <10 mm. A complete response of a non-target lesion includes disappearance of all non-target lesions and normalization of tumor marker level (all lymph nodes must be non-pathological in size (<10 mm short axis)). If tumor markers are initially above the upper normal limit, they need to normalize for a patient to be considered in complete clinical response of a nontarget lesion. The duration of overall CR is measured from the time measurement criteria are first met for CR until the first date that progressive disease is objectively documented, or death due to any cause. Participants without events reported are censored at the last disease evaluation.

As used herein, the term “stable disease” or “SD” means neither sufficient shrinkage to qualify for PR nor sufficient increase to qualify for PD, taking as reference the smallest sum diameters while on study. Duration of stable disease is measured from the start of the treatment until the criteria for progression are met, taking as reference the smallest measurements recorded since the treatment started, including the baseline measurements.

As used herein, the term “progressive disease” or “PD” when refers to a target lesion means at least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progressions). When progressive disease or PD refers to a non-target lesion, it means the appearance of one or more new lesions and/or unequivocal progression of existing non-target lesions. Unequivocal progression should not normally trump target lesion status. It must be representative of overall disease status change, not a single lesion increase.

As used herein, the term “best overall response” means the best response recorded from the start of the treatment until disease progression/recurrence (taking as reference for progressive disease the smallest measurements recorded since the treatment started). The patient's best response assignment depends on the achievement of both measurement and confirmation criteria. The duration of an overall response is measured from the time measurement criteria are met for CR or PR (whichever is first recorded) until the first date that recurrent or progressive disease is objectively documented (taking as reference for progressive disease the smallest measurements recorded since the treatment started, or death due to any cause. Participants without events reported are censored at the last disease evaluation).

As used herein, the term “DLT rate” means dose-limiting toxicity rate.

As used herein, the term “IC₅₀” means inhibitory drug concentration that produces 50% of the maximal effect.

As used herein, the term “SEM” means standard error of mean.

As used herein, the term “AUC_{(x-y)}” means area under the curve, wherein “x” is the starting time in hours and “y” is the ending time in hours.

As used herein, the term “C_{avg}” means average concentration. As used herein, the term “Cₘₐₓ” means maximum concentration.

As used herein, the term “ANC” means absolute neutrophil count.

As used herein, the term “CT” means computed tomography.

As used herein, the term “ctDNA” means circulating tumor DNA.

As used herein, the term “MRI” means magnetic resonance imaging.

As used herein, the term “PK” means pharmacokinetic.

As used herein, the term “PBMC” means peripheral blood mononuclear cells.

As used herein, the term “tolerable” means a dose level where ≤⅙ participants have experienced a DLT, or the dose level that is declared the RP2D.

As used herein, the term “adverse event” or “AE” means an untoward medical occurrence in a subject administered a medicinal product that does not necessarily have a causal relationship with this treatment. An AE can be an unfavorable and unintended sign (including an abnormal laboratory finding), symptom, or disease temporally associated with the use of an investigational product, whether or not related to the investigational medicinal product. An adverse events may include worsening or exacerbation of the disease under study; worsening or exacerbation of pre-existing conditions or events; intercurrent illnesses; or drug interactions. Anticipated fluctuations of pre-existing conditions that do not represent a clinically significant exacerbation or worsening are not considered AEs. Surgical procedures are not adverse events; they are therapeutic measures for conditions that require surgery. The condition, provided it develops or is a worsening of a pre-existing condition for which the surgery is required, is the AE. Disease progression is an efficacy endpoint and is not an AE. A clinical event in the setting of disease progression would be considered an AE if it could not be unequivocally attributed to or consistent with expected disease progression.

As used herein, the term “expected adverse event” means an adverse event that are listed or characterized in the current adverse event list, the Package Insert, the Investigator Brochure or is included in the informed consent document as a potential risk.

As used herein, the term “unexpected adverse event” means an adverse event that is not listed in the Package Insert (P.I.) or current Investigator Brochure (I.B.) or not identified. This includes adverse events for which the specificity or severity is not consistent with the description in the P.I. or I.B. For example, under this definition, hepatic necrosis would be unexpected.

As used herein, the term “severe adverse event” or “SAE” means an AE that (1) results in death (i.e., the AE actually causes or leads to death); (2) is life threatening (i.e., the AE, in the view of the investigator, places the subject at immediate risk of death, but does not include an AE that, had it occurred in a more severe form, might have caused death); (3) requires or prolongs inpatient hospitalization; (4) results in persistent or significant disability/incapacity (i.e., the AE results in substantial disruption of the subject's ability to conduct normal life functions); or (5) results in a congenital anomaly/birth defect in a neonate/infant born to a mother exposed to the IMP.

As used herein, the term “definite AE” means the AE is clearly related to the study treatment.

As used herein, the term “probable AE” means the AE is likely related to the study treatment.

As used herein, the term “possible AE” means the AE may be related to the study treatment.

As used herein, the term “unlikely AE” means the AE is doubtfully related to the study treatment.

As used herein, the term “unrelated AE” means the AE is clearly not related to the study treatment.

As used herein, the term “expected disease progression” means an event that is unequivocally related to disease progression, and that the clinical course is consistent with what would be expected for the patient's disease.

As used herein, the term “measurable lesion” means a lesion that can be accurately measured in at least one dimension (longest diameter to be recorded) as ≥20 mm by chest x-ray or ≥10 mm with CT scan, MRI, or calipers by clinical exam. Tumor lesions that are situated in a previously irradiated area might or might not be considered measurable. Cystic lesion thought to represent cystic metastases are measurable lesions if they meet the definition of measurability described above. However, they are target lesions if non-cystic lesions are also present in the same participant. Clinical lesions are measurable when they are superficial (e.g., skin nodules and palpable lymph nodes) and ≥10 mm in diameter as assessed using calipers (e.g., skin nodules).

As used herein, the term “malignant lymph node” means a pathologically enlarged and measurable lymph node with ≥15 mm in short axis when assessed by CT scan.

As used herein, the term “non-measurable disease” means a small lesion (or a site of disease) where the longest diameter <10 mm or pathological lymph nodes with ≥10 to <15 mm short axis. Bone lesions, leptomeningeal disease, ascites, pleural/pericardial effusions, lymphangitis cutis/pulmonitis, inflammatory breast disease, abdominal masses (not followed by CT or MRI), and cystic lesions are examples of non-measurable disease. Cystic lesions that meet the criteria for radiographically defined simple cysts are not malignant lesions (neither measurable nor non-measurable) and are simple cysts.

As used herein, the term “target lesion” means all measurable lesions up to a maximum of 2 lesions per organ and 5 lesions in total, that is representative of all involved organs. Target lesions are selected on the basis of their size (lesions with the longest diameter), be representative of all involved organs, but in addition should be those that lend themselves to reproducible repeated measurements. When the largest lesion does not lend itself to reproducible measurement, the next largest lesion that can be measured reproducibly is the target lesion.

As used herein, the term “non-target lesions” means all lesions (or sites of disease) that are not target lesions. Non-target lesions include any measurable lesions over and above the 5 target lesions.

As used herein, the term “overall survival” or “OS” means the time from randomization (or registration) to death due to any cause, or censored at date last known alive.

As used herein, the term “progression-free survival” or “PFS” means the time from randomization (or registration) to the earlier of progression or death due to any cause. Participants alive without disease progression are censored at date of last disease evaluation.

As used herein, the term “time to progression” or “TTP” means the time from randomization (or registration) to progression, or censored at date of last disease evaluation for those without progression reported.

As used herein, the term “metastasis” refers to a process in which cancer cells travel from one organ or tissue to another non-adjacent organ or tissue. Cancer cells can spread to tissues and organs of a subject, and conversely, cancer cells from other organs or tissue can invade or metastasize to the lung. Cancerous cells may invade or metastasize to any other organ or tissue of the body. The term “invasion”, in some embodiments, refers to the spread of cancerous cells to adjacent surrounding tissues.

As used herein, the term “about” can mean plus or minus 5% of the provided value.

### Methods and Compositions for Treating RAS^{WT} Colorectal Cancer

Polo-like kinase 1 (PLK1) is a serine/threonine protein kinase and a key regulator of the cell cycle. PLK1 is overexpressed in colorectal cancer (CRC) and this overexpression is associated with poor prognosis. PLK1 inhibition has been shown to sensitize non-small lung cancer to EGFRi in preclinical models. Onvansertib is an oral, small molecule, selective inhibitor of PLK1 and has shown robust antitumor activity in combination with irinotecan and bevacizumab in RAS-mutant CRC xenograft models. Onvansertib is currently under clinical development in combination with chemotherapy+bevacizumab for RAS-mutant mCRC (NCT03829410 and NCT06106308).

Provided herein are methods and data related to the efficacy of onvansertib as monotherapy and in combination with cetuximab in RAS^{WT} CRC patient-derived xenograft (PDX) models, sensitive or resistant to cetuximab.

Colorectal cancer is a heterogeneous disease complicated by the common occurrence of several molecular alterations comprising the epidermal growth factor receptor (EGFR) pathway, including mutations in Kirsten rat sarcoma (KRAS), neuroblastoma RAS viral oncogene homolog (NRAS), and v-raf murine sarcoma viral oncogene homolog B1 (BRAF [V600E]), and in the human epidermal growth factor receptor 2 (HER2) and MET receptors. Other molecular alterations include DNA damage repair mechanisms and rare kinase fusions. Moreover, tumor sidedness is associated with distinct clinical and biological characteristics. Right-sided CRC is more common in women, and associated with Lynch syndrome, mitogen-activated protein kinase (MAPK)-signaling, high microsatellite instability (MSI-H), deficiency of mismatch repair genes, CpG island methylation, and KRAS and BRAF mutations. Left-sided CRC is more common in men, and associated with familial adenomatous polyposis syndrome, wingless-related integration site (Wnt) and EGFR signaling, chromosomal instability, v-erb-b2 erythroblastic leukemia viral oncogene homolog 1 (ERBB1) and ERBB2 amplifications, adenomatous polyposis coli (APC), p53 and NRAS mutations. These alterations represent oncogenic drivers that may coexist in the same tumor with other primary and acquired alterations via a clonal selection process.

Advances have been made in the therapy of CRC in targeted subgroups with specific mutational profiles. However, resistance to these targeted agents as well as standard chemotherapies based on specific molecular alterations have confounded treatment. Increasingly, enhanced knowledge about tumor biology is driving therapeutic decision-making. Optimal combinations and sequencing of these agents is continuing to evolve. Known biologic drugs that are active against mCRC include agents targeting vascular endothelial growth factors (VEGFs) and their receptors (VEGFRs), EGFR, BRAF V600E, HER2, immunotherapy using immune checkpoint inhibitors, and tropomyosin receptor kinase (TRK) inhibitors.

Anti-EGFR antibodies (in some embodiments, in combination with chemotherapy) such as cetuximab and panitumumab have significantly improved prognosis in RAS wild-type patients (RAS^{WT}). In addition, patients with MSI-H have demonstrated impressive responses to checkpoint inhibitors such as the anti-programmed cell death-1 (PD-1) agent pembrolizumab.

EGFR (also known as ErbB-1 or HER-1) inhibitors are medicines that bind to certain parts of the EGFR and slow down or stop cell growth. EGFR is a protein that is found on the surface of some cells that causes cells to divide when epidermal growth factor binds to it. EGFR is found at abnormally high levels in cancer cells, and EGFR activation appears to be important in tumor growth and progression. Some types of cancers show mutations in their EGFRs, which may cause unregulated cell division through continual or abnormal activation of the EGFR. EGFR inhibitors can be classified as either: tyrosine kinase inhibitors (TKI) (e.g., erlotinib and gefitinib) or as monoclonal antibodies (e.g., cetuximab and necitumumab). TKIs bind to the tyrosine kinase domain in the epidermal growth factor receptor and stop the activity of the EGFR. Monoclonal antibodies bind to the extracellular component of the EGFR and prevent epidermal growth factor from binding to its own receptor, therefore preventing cell division.

EGFR inhibitors may be used in the treatment of cancers that are caused by EGFR up-regulation, such as non-small-cell lung cancer, pancreatic cancer, breast cancer, and colon cancer. However, resistance to these drugs has limited their effectiveness. Pre-clinical data has shown that PLK1 inhibition, without being bound by any particular theory, results in degradation of EGFR via E3-ubiquitin ligase pathway.

In some embodiments, the RAS^{WT} cancer is refractory colorectal cancer that does not respond to treatment such as VEGF inhibitors or EGFR inhibitors. Refractory colorectal cancer can also be referred to as “resistant cancer.” The RAS^{WT} colorectal cancer may be resistant at the beginning of the treatment (intrinsic resistance) or it may become resistant during treatment (acquired or induced resistance). In some embodiments, the RAS^{WT} colorectal cancer is resistant or does not respond to EGFR inhibitor (EGFRi) therapies such as antibodies that bind to EGFR (e.g., cetuximab or panitumumab) or tyrosine kinase inhibitors (e.g., regorafenib). For example, the subject having RAS^{WT} colorectal cancer can have stable or progressive disease following EGFRi therapies. In some embodiments, the subject having RAS^{WT} colorectal cancer has resistance to cetuximab, panitumumab, or both. In some embodiments, the RAS^{WT} colorectal cancer is also wild-type for BRAF (e.g., is RAS/BRAF^{WT}). In some embodiments, the RAS^{WT} or RAS/BRAF^{WT} colorectal cancer is left-sided. In some embodiments, the RAS^{WT} or RAS/BRAF^{WT} colorectal cancer is right-sided. The distinction between right-sided and left-sided colon is based on their embryological origins. The cecum, appendix, ascending colon, hepatic flexure, and proximal two thirds of the transverse colon have originated from the midgut, whereas distal one third of the transverse colon, splenic flexure, sigmoid colon, descending colon and rectum have originated from the hindgut. Hence, the right-sided CRC (RCRC) tumors arise from ascending colon, and proximal two thirds of the transverse colon and the left-sided CRC (LCRC) tumors arise from the descending and sigmoid colon, and distal one third of the transverse colon. Besides the difference in their embryological origin, these sideness of tumors is associated with differences in histology and pathology of the tumors as well as clinical outcomes. For example, while right-sided tumors show sessile serrated adenomas or mucinous adenocarcinomas, left-sided tumors show tubular, villous and typical adenocarcinomas. Since left-sided tumors have polypoid morphology, it is easier to detect them with colonoscopy in the early stages of carcinogenesis. RCRC has flat morphology that is difficult to detect. Furthermore, RCRC patients tend to have advanced and bigger tumors, which are often poorly differentiated. The genomic make-up of RCRC and LCRC are totally different from each other. While RCRC patients tend to have more microsatellite instability-high (MSI-high) tumors, LCRC patients tend to have chromosomal instability-high (CIN-high) tumors. There are also reports on the differences of efficacy of first-line therapies for RCRC and LCRC. For instance, cetuximab and panitumumab are the most effective treatments for left-sided mCRC, while bevacizumab is a more optimal choice for right-sided mCRC.

The RAS^{WT} colorectal cancer and/or tumor can be a cancer and/or tumor, having abnormal alterations to PLK1 gene or protein. It has been identified that polo-like kinase 1 (PLK1) is an important gene for growth and survival of colon cancer cells with unstable genome. For example, the abnormal alterations can include one or more PLK1 alterations and/or PLK1 aberrant activation such as copy number alteration (CNA), single-nucleotide variation (SNV), and gene rearrangement or fusions. Non-limiting exemplary cancer and/or tumor with PLK1 alterations include cancer with PLK1 gene or protein amplification, PLK1 gene or protein modification, PLK1 gene deletion, PLK1 gene or protein overexpression, elevated PLK1 gene or protein expression, and/or a combination thereof. In some embodiments, the cancer and/or tumor can be a PLK1-amplified cancer in which PLK1 gene and/or protein is amplified, for example, as a result of gene duplication and/or aberrant gene transcriptional control. For example, the cancer with PLK1 amplification can be a cancer with higher PLK1 mRNA and/or protein levels as compared to healthy tissues. In heterogenous cancer types, the RAS^{WT} colorectal cancer/or tumor can include a subtype that has an abnormal high expression of PLK1 gene and/or protein. In some embodiments, the RAS^{WT} colorectal cancer and/or tumor with amplified PLK1 can be node-positive tumors, aggressive tumors and/or invasive tumors. In some embodiments, the RAS^{WT} colorectal cancer and/or tumor with amplified PLK1 can have a shorter disease-free survival as compared to cancer and/or tumor with normal levels of PLK1. In some embodiments, the RAS^{WT} colorectal cancer and/or tumor exhibits a high relapse and/or resistance to traditional and/or mono-therapies, such as hormone therapy, chemotherapy and/or radiotherapy.

In some embodiments, the colorectal cancer is resistant to or does not respond effectively to (e.g., has stable or progressive disease) mono-treatment with an EGFR inhibitor (e.g., an antibody capable of binding to EGFR). Exemplary EGFR inhibitors include: erlotinib, lapatinib, AZD8931, WZ4002, panitumumab, vandetanib, icotinib, afatinib, brigatinib, CO-1688, AZD-4769, poziotinib, CUDC-101, S-222611, AC-480, imgatuzumab, sapitinib, TAS-2913, theiiatinib, XGFR-2421, HM-61713B, epitinib, NRC-2694, MLBS-42, JRP-890, cetuximab, AL-6802, TAK-285, BGB-102, AEE788, gefitinib, DMS-3008, TX-2036, KI-6783, and KI-6896.

### Polo-Like Kinase Inhibitors

Disclosed herein include methods of treating RAS wild type (RAS^{WT}) colorectal cancer. In some embodiments, the method comprises: administering a polo-like kinase 1 (PLK1) inhibitor to a subject with a RAS^{WT} colorectal cancer, thereby inhibiting or reducing progression of the RAS^{WT} colorectal cancer in the subject.

Polo-like kinases (PLK) are a family of five highly conserved serine/threonine protein kinases. PLK1 is a master regulator of mitosis and is involved in several steps of the cell cycle, including mitosis entry, centrosome maturation, bipolar spindle formation, chromosome separation, and cytokinesis. It is also critical for the entry and progression through mitosis, regulates progression of cells through the G2 phase of the cell cycle by phosphorylating forkhead box protein M1 (FOXM1), which then regulates the expression of cyclins and other genes necessary for cells to progress through the cell cycle. PLK1 has been shown to be overexpressed in solid tumors and hematologic malignancies, including colorectal cancers. Patients with high PLK1 expression typically have poor prognosis. Several PLK inhibitors have been studied in clinical trials. In the early pre-clinical development of PLK1 targeted drugs, cancer cells with TP53 (e.g., p53) mutation (mutp53) were more responsive and had lower IC₅₀ than cell lines with wild type (wtp53), which are consistent with the lack of checkpoint control and genomic instability associated with mutp53 and increases the importance of PLK1 function for progression through G2 and M phases of the cell cycle. The pyruvate dehydrogenases kinase 1 (PDK1), PLK1, and MYC have also been suggested to be important in driving the expression of a set of genes associated with cancer stem cell self-renewal.

In a randomized phase II study of patients with acute myeloid leukemia (AML) who were treatment naïve yet unsuitable for induction therapy, the pan-PLK inhibitor, volasertib (BI6727), administered intravenously in combination with low-dose cytarabine (LDAC) showed a significant increase in overall survival (OS) when compared with LDAC alone. A subsequent randomized phase III study identified no benefit of the combination and described an increased risk of severe infections. PLK1 facilitates HR during Double Strand DNA Break (DSB) Repair. PLK1 phosphorylates Rad51 and BRCA1, facilitating their recruitment to DSB sites and thereby HR-mediated DNA repair.

Onvansertib (also known as PCM-075, NMS-1286937, NMS-937, “compound of formula (I)” in U.S. Pat. No. 8,927,530; IUPAC name 1-(2-hydroxyethyl)-8-{[5-(4-methylpiperazin-1-yl)-2-(trifluoromethoxy)phenyl]amino}-4,5-dihydro-1H-pyrazolo[4,3-h]quinazoline-3-carboxamide), or a pharmaceutically acceptable salt, is a selective ATP-competitive PLK1 inhibitor. Onvansertib can be formulated, e.g., with an additive such as free base, lactose monohydrate, pregelatinized starch and glyceryl beneate. In some embodiments, the onvansertib is formulated for oral administration, such as in a hard gelatin capsule.

Biochemical assays demonstrated high specificity of onvansertib for PLK1 among a panel of 296 kinases, including other PLK members. Onvansertib has potent in vitro and in vivo antitumor activity in models of both solid and hematologic malignancies. Onvansertib is the first PLK1 specific ATP competitive inhibitor administered by oral route to enter clinical trials with proven antitumor activity in different preclinical models. Onvansertib has shown a promising safety profile in a phase 1 clinical trial as single agent.

Onvansertib also inhibited cell proliferation at nanomolar concentrations in AML cell lines and tumor growth in xenograft models of AML. In addition, onvansertib significantly increased cytarabine antitumor activity in disseminated models of AML.

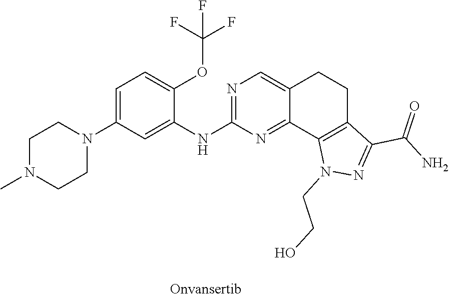 [SMILES: CN1CCN(c2ccc(OC(F)(F)F)c(Nc3ncc4c(n3)-c3c(c(C(N)=O)nn3CCO)CC4)c2)CC1]

Onvansertib shows high potency in proliferation assays having low nanomolar activity on a large number of cell lines, both from solid as well as hematologic tumors. Onvansertib has a relative short half-life of 24 h and is highly potent against the PLK1 enzyme ([IC₅₀]=2 nM). In comparison, low or no activity was observed on a panel of 63 kinases (IC₅₀>500 nM), including the PLK members PLK2 and PLK3 (IC₅₀>10 μM). Onvansertib potently causes a mitotic cell-cycle arrest followed by apoptosis in cancer cell lines and inhibits xenograft tumor growth with a clear PLK1-related mechanism of action at well tolerated doses in mice after oral administration. Onvansertib has favorable pharmacologic parameters and good oral bioavailability in rodent and nonrodent species, as well as proven antitumor activity in different nonclinical models using a variety of dosing regimens, which may potentially provide a high degree of flexibility in dosing schedules, warranting investigation in clinical settings. Onvansertib has several advantages over volasertib (BI6727, another PLK1 inhibitor), including a higher degree of potency and specificity for the PLK1 isozyme, and oral bioavailability. In addition, onvansertib has proven antitumor activity in different nonclinical models using a variety of dosing regimens, which can provide flexibility in dosing schedules, and therefore, warrants investigation in clinical settings.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window. Pharmacodynamics and biomarker studies, including baseline genomic profiling, serial monitoring of mutant allele fractions in plasma, and the extent of PLK1 inhibition in circulating blasts, have been performed to identify biomarkers associated with clinical response and are described in PCT Application No. PCT/US2021/013287, the content of which is incorporated herein by reference in its entirety.

The major metabolic pathways found in the different animal species were N-oxidation of the N methyl-piperazine ring to give N-oxide M2 and hydroxylation on an aliphatic carbon atom of the methylene bridge of the pyrazoloquinazoline moiety to give metabolite M1. Qualitatively, no marked differences in the metabolism of onvansertib were observed between species and, quantitatively, some differences were observed cross-species.

The potential inhibitory capacity of onvansertib towards the major human cytochrome P450 (CYP) isoforms that are responsible for hepatic drug metabolism in human (CYP1A2, CYP2C8, CYP2C9, CYP2C19, CYP2D6, and CYP3A4) was investigated using human liver microsomes. Onvansertib was able to inhibit the metabolic activities of CYP2C8, CYP2C9, CYP2C19, CYP2D6, and CYP3A4 isoforms to different extents, with 50% inhibitory concentration (IC50) values ranging from 20 μM to 66 μM. No significant inhibitory effects against CYP1A2 were detected. Considering that the concentrations relevant to achieve significant anti-tumoral activity of the compound in mice were in the order of 1 μM, the likelihood that onvansertib would show clinically relevant metabolic drug-drug interactions is considered low.

In a Phase 1 safety study with onvansertib that has been completed in adult patients with advanced/metastatic solid tumors at a single study site in the US, first cycle dose-limiting toxicities (DLTs) and the maximum tolerated dose (MTD) of onvansertib administered orally for 5 consecutive days every 3 weeks (i.e., a 21 day treatment cycle) was conducted. Safety profile of onvansertib, to determine the pharmacokinetics (PK) of onvansertib in plasma (at the MTD), and to document any antitumor activity has been determined. In one study, a total of 21 patients were enrolled, and 19 patients were treated. No DLTs occurred at the first 3 dose levels (doses of 6, 12, and 24 mg/m²/day). At the subsequent dose level (dose of 48 mg/m²/day), 2 of 3 patients developed DLTs. An intermediate dose level of 36 mg/m²/day was investigated. At the intermediate dose level, 4 patients were treated and 2 DLTs were observed. After further cohort expansion, the MTD was determined to be 24 mg/m²/day. The best observed treatment response was stable disease (SD); SD occurred in 5 of the 16 evaluable patients. The study identified thrombocytopenia and neutropenia as the primary toxicities; this is consistent with the expected mechanism of action of onvansertib and with results from the preclinical studies. These hematologic toxicities were reversible, with recovery usually occurring within 3 weeks. No other clinically relevant safety findings emerged with treatment with onvansertib as a single agent. Other mechanism-related, possibly expected events such as gastrointestinal disorders, mucositis, and alopecia were not observed, confirming that with this schedule, the bone marrow is the most sensitive target of onvansertib in humans.

In some embodiments, a PLK-1 inhibitor can be used (e.g., as a monotherapy) for treatment of colorectal cancer (e.g., RAS^{WT} colorectal cancer) in a subject that is or has become resistant to an EGFR inhibitor. In some embodiments, the method comprises administration of PLK-1 inhibitor alone without the administration of additional compositions or therapies for treating cancer. In some embodiments, the method does not comprise administering irinotecan, bevacizumab, or both to the subject with the RAS^{WT} colorectal cancer.

The subject with the RAS^{WT} colorectal cancer can be resistant to or does not respond effectively to an epidermal growth factor receptor (EGFR) inhibitor. The subject with the RAS^{WT} colorectal cancer can be resistant to treatment with the EGFR inhibitor or can have stable or progressive disease following treatment with the EGFR inhibitor. The subject with RAS^{WT} colorectal cancer can be resistant to cetuximab, panitumumab, or both. In some embodiments, the subject with the RAS^{WT} colorectal cancer has stable or progressive disease following treatment with cetuximab, panitumumab, or both. The resistance can be acquired resistance or intrinsic resistance.

In some embodiments, the subject with the RAS^{WT} colorectal cancer has received a prior EGFR inhibitor therapy. In some embodiments, the subject with the RAS^{WT} colorectal cancer did not respond to the treatment with the prior EGFR inhibitor therapy. In some embodiments, the subject with the RAS^{WT} colorectal cancer has stable or progressive disease following the treatment with the prior EGFR inhibitor therapy. In some embodiments, the subject with the RAS^{WT} colorectal cancer is known to be resistant to the EGFR inhibitor therapy.

The PLK1 inhibitor can be administered to the subject in a cycle of 7 days, 14 days, 21 days, 28 days, 35 days, 42 days, or 49 days. The PLK1 inhibitor can be administered to the subject daily. Each cycle of treatment can be at least about 14 days. Each cycle of treatment can be from about 14 days to about 28 days. The PLK1 inhibitor can be administered on at least five days, at least ten days, or at least fifteen days in a cycle. In some embodiments, the PLK1 inhibitor is not administered on at least one day, at least three days, or at least seven days in a cycle. The days with PLK1 inhibitor can be continuous or separated by certain intervals. For example, PLK1 inhibitor can be administered on the first continuous ten days or on Days 1-5 and 15-19 of a 28-day circle. In some embodiments, the subject undergoes at least two cycles of the administration of the PLK1 inhibitor.

The EGFR inhibitor (e.g., that the RAS^{WT} colorectal cancer is resistant to) can be erlotinib, lapatinib, AZD8931, WZ4002, panitumumab, vandetanib, icotinib, afatinib, brigatinib, CO-1688, AZD-4769, poziotinib, CUDC-101, S-222611, AC-480, imgatuzumab, sapitinib, TAS-2913, theiiatinib, XGFR-2421, HM-61713B, epitinib, NRC-2694, MLBS-42, JRP-890, cetuximab, AL-6802, TAK-285, BGB-102, AEE788, gefitinib, DMS-3008, TX-2036, KI-6783, KI-6896; or a pharmaceutically acceptable salt, hydrate, solvate or prodrug thereof; or any combination thereof. The EGFR inhibitor can be cetuximab or panitumumab.

The PLK1 inhibitor can be onvansertib (NMS-P937), BI2536, volasertib (BI 6727), GSK461364, adavosertib (AZD1775), CYC140, HMN-176, HMN-214, rigosertib (ON-01910), MLN0905, TKM-080301, TAK-960, GTPL10072, Ro3280; or a pharmaceutically acceptable salt, hydrate, solvate or prodrug thereof; or any combination thereof. The PLK1 inhibitor can be onvansertib.

### Combination Therapy

Also provided herein are combination therapies. Disclosed herein include methods of treating RAS wild type (RAS^{WT}) colorectal cancer. In some embodiments, the method comprises: administering a PLK1 inhibitor and a first EGFR inhibitor to a subject with the RAS^{WT} colorectal cancer, thereby inhibiting or reducing progression of the RAS^{WT} colorectal cancer in the subject.

In some embodiments, the inhibition or reduction of cancer progression by the combination is not merely additive, but is enhanced or synergistic (that is, the inhibition is greater than the combined inhibition of progression caused by the PLK1 inhibitor and the first EGFR inhibitor alone). The enhanced or synergistic efficacy or inhibition of any combination of the PLK1 inhibitor and the first EGFR inhibitor of the present disclosure can be different in different embodiments. In some embodiments, the enhanced or synergistic efficacy or inhibition of any combination of a the PLK1 inhibitor and an EGFR inhibitor of the present disclosure is, is about, is at least, is at least about, is at most, or is at most about, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values, higher than the combined inhibition of progression caused by the PLK1 inhibitor and the EGFR inhibitor (e.g., the first EGFR inhibitor).

The molar ratio of the PLK-1 inhibitor (e.g., onvansertib) to the first EGFR inhibitor (e.g., cetuximab) can be, for example, about 1:200, 1:100, 1:90, 1:80, 1:70, 1:60, 1:50, 1:40, 1:30, 1:20, 1:10, 1:1, 10:1, 20:1, 30:1, 40:1, 50:1, 100:1, 1000:1, or 2000:1, or a number or a range between any two of these values. In some embodiments, the enhanced or synergistic efficacy or inhibition of cancer progression caused by a combination of the PLK-1 inhibitor (e.g., onvansertib) with the first EGFR inhibitor (e.g., cetuximab) is, is about, is at least, is at least about, is at most, or is at most about 20%, 30%, 40%, 50%, 60%, 70%, 80%, 90%, 100%, 110%, 120%, 130%, 140%, 150%, 160%, 170%, 180%, 190%, 200%, 250%, 300%, or a number or a range between any two of these values, higher than the combined inhibition of progression caused by the PLK-1 inhibitor (e.g., onvansertib) alone plus the first EGFR inhibitor (e.g., cetuximab) alone. For example, a combination of the PLK-1 inhibitor (e.g., onvansertib) and the first EGFR inhibitor (e.g., cetuximab) can cause a 50%, 60%, 70%, 80%, 90%, or more, inhibition of cancer progression (cancer cell viability of 50%, 40%, 30%, 20%, 10%, or less), whereas under the same conditions the combined inhibition of the PLK-1 inhibitor (e.g., onvansertib) alone plus the first EGFR inhibitor (e.g., cetuximab) alone can be 10%, 20%, 25%, 30%, or less) inhibition of cancer progression (cancer cell viability of 90%, 80%, 75%, 70%, or more). Thus, the enhanced or synergistic efficacy or inhibition of cancer progression caused by the combination of the PLK-1 inhibitor (e.g., onvansertib) and the first EGFR inhibitor (e.g., cetuximab) is, for example, 50%, 60%, 70%, 80%, 90%, 100%, or more higher than the combined inhibition of progression caused by the PLK-1 inhibitor (e.g., onvansertib) alone plus the first EGFR inhibitor (e.g., cetuximab) alone. In some embodiments, the PLK-1 inhibitor is onvansertib and the first EGFR inhibitor is cetuximab or panitumumab.

In some embodiments, the subject with the RAS^{WT} colorectal cancer is resistant to or does not respond effectively to a second EGFR inhibitor. In some embodiments, the subject with the RAS^{WT} colorectal cancer is resistant to treatment with the second EGFR inhibitor or has stable or progressive disease following treatment with the second EGFR inhibitor. In some embodiments, the subject with RAS^{WT} colorectal cancer is resistant to cetuximab, panitumumab, or both or has stable or progressive disease following treatment with cetuximab, panitumumab, or both. The resistance can be acquired resistance or intrinsic resistance. The first and second EGFR inhibitors can be the same. The first and second EGFR inhibitors can be different.

In some embodiments, the subject with the RAS^{WT} colorectal cancer has received a prior EGFR inhibitor therapy (e.g., the second EGFR inhibitor). The EGFR inhibitor can be the second EGFR inhibitor. In some embodiments, the subject with the RAS^{WT} colorectal cancer did not respond to the treatment with the prior EGFR inhibitor therapy. In some embodiments, the subject with the RAS^{WT} colorectal cancer has stable or progressive disease following the treatment with the prior EGFR inhibitor therapy. In some embodiments, the subject with the RAS^{WT} colorectal cancer is known to be resistant to the second EGFR inhibitor.

In some embodiments, administering to the subject having RAS^{WT} colorectal cancer that is resistant to or does not respond effectively to a second EGFR inhibitor in combination with a PLK-1 inhibitor, can unexpectedly enhance the therapeutic effect in treating the RAS^{WT} colorectal cancer. When combined, surprisingly, the two inhibitors (e.g., a PLK-1 inhibitor and an EGFR inhibitor) can obtain complete cell division inhibition and resulting tumor regression and cancer survival rate/duration. The combination can be surprisingly synergistic (i.e., more than additive, superior to the cumulated anti-tumor efficacy caused by the EGFR inhibitor alone).

In some embodiments, the combination treatment with the PLK-1 inhibitor and the first EGFR inhibitor can be administered at the same dose as single treatment with the PLK-1 inhibitor and the first EGFR inhibitor. As can be appreciated by one of skill in the art, the amount of co-administration of the PLK-1 inhibitor and the first EGFR inhibitor, and the timing of co-administration, can depend on the type (species, gender, age, weight, etc.) and condition of the subject being treated and the severity of the disease or condition being treated. The PLK-1 inhibitor and the first EGFR inhibitor can formulated into a single pharmaceutical composition, or two separate pharmaceutical compositions. The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions.

The PLK-1 inhibitor and the first EGFR inhibitor can be administered by any suitable routes, including but not limited to oral, topical (including buccal and sublingual), rectal, vaginal, transdermal, parenteral, subcutaneous, intraperitoneal, intrapulmonary, intradermal, intrathecal, epidural, and intranasal administration. Parenteral administration (e.g., injection) can include intramuscular, intravenous, intraarterial, intraperitoneal, or subcutaneous administration. In some embodiments, the first EGFR inhibitor (e.g., cetuximab) can be, for example, administered by intravenous infusion (e.g., over about 30 minutes) and the PLK-1 inhibitor (e.g., onvansertib) can be, for example, administered orally.

Methods, compositions, kits and systems disclosed herein can be applied to different types of subjects. For example, the subject can be a subject receiving a cancer treatment, a subject at cancer remission, a subject has received one or more cancer treatments, or a subject suspected of having cancer. The subject can have a stage I cancer, a stage II cancer, a stage III cancer, and/or a stage IV cancer.

### Dosing and Pharmacokinetics

The treatment described herein can comprise administration of a PLK-1 inhibitor (e.g., onvansertib) for a desired duration in one or more cycles of treatment. The treatment can comprise administration of a PLK-1 inhibitor (e.g., onvansertib) and a first EGFR inhibitor for a desired duration in one or more cycles of treatment.

Daily or weekly administration of an EGFR inhibitor such as a tyrosine kinase inhibitor (TKI) can be at, or be about, 0.01 mg, 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 5 mg, 10 mg, 20 mg, 50 mg, 100 mg, 200 mg, 300 mg, 400 mg, 500 mg, 600 mg, 700 mg, 800 mg, 900 mg, 1000 mg, 1100 mg, 1200 mg, or a number or a range between any two of these values. The daily or weekly dose of the first EGFR inhibitor can be adjusted (e.g., increased or decreased with the range) during the treatment of the subject. The daily or weekly administration of the first EGFR inhibitor can be at different amounts on different days or during different weeks. For example, the treatment can comprise daily or weekly administration of the first EGFR inhibitor at 0.1 mg to 20 mg during week 1, 0.25 mg to 50 mg during week 2, 0.5 mg to 100 mg during week 3, 1 mg to 200 mg during week 4, and 2 mg to 400 mg during week 5 and beyond. For example, the treatment can comprise daily or weekly administration of the first EGFR inhibitor at 0.1 mg to 100 mg on day 1, 0.2 mg to 200 mg on day 2, 0.4 mg to 400 mg on day 3, and 0.4 mg to 400 mg or 0.6 mg to 600 mg on day 4 and beyond. For example, the first EGFR inhibitor can be a TKI and is administered at a daily or weekly dose of about 10 mg, 20 mg, 30 mg, 40 mg, 50 mg, 60 mg, 70 mg, 80 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 1000 mg, or a number or a range between any two of these values.

In some embodiments, the first EGFR inhibitor can be administered daily or weekly at a drug/body surface area unit dose of about 15 mg/m² to about 500 mg/m². For example, in some embodiments, the first EGFR inhibitor is an antibody (e.g., cetuximab or panitumumab) and can be administered at, or at about 5 mg/m², 6 mg/m², 7 mg/m², 8 mg/m², 9 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 205 mg/m², 210 mg/m², 215 mg/m², 220 mg/m², 225 mg/m², 230 mg/m², 235 mg/m², 240 mg/m², 245 mg/m², 250 mg/m², 300 mg/m², 350 mg/m², 400 mg/m², 450 mg/m², 500 mg/m², or a number or a range between any two of these values.

In some embodiments, the first EGFR inhibitor can be administered daily or weekly at a drug/body surface area unit dose of about 1 mg/kg to about 500 mg/kg. For example, in some embodiments, the first EGFR inhibitor is an antibody (e.g., cetuximab or panitumumab) and can be administered at, or at about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, 205 mg/kg, 210 mg/kg, 215 mg/kg, 220 mg/kg, 225 mg/kg, 230 mg/kg, 235 mg/kg, 240 mg/kg, 245 mg/kg, 250 mg/kg, 300 mg/kg, 350 mg/kg, 400 mg/kg, 450 mg/kg, 500 mg/kg, or a number or a range between any two of these values.

Each cycle of treatment/administration can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. In some embodiments, the first EGFR inhibitor is administered daily, semi-weekly, or weekly for three weeks in a 28 day cycle. In exemplary embodiments, the first EGFR inhibitor is administered for 1 to 10 cycles, for example, 1 to 9 cycles, 1 to 8 cycles, 1 to 7 cycles, 1 to 6 cycles, 1 to 5 cycles, 1 to 4 cycles, 1 to 3 cycles, 1 to 2 cycles, or 1 cycle. The administration of the first EGFR inhibitor can be daily or weekly and/or with break(s) between the administrations. The break can be, for example, 1 day, 2 days, 3 days, 4 days, 5 days, 6 days, 7 days, 8 days, 9 days, 10 days, 11 days, 12 days, 13 days, 14 days, or more. In some embodiments, the breaks can be 6 days and/or 13 days. In some embodiments, the daily or weekly dose of the first EGFR inhibitor can be adjusted (e.g., increased or decreased with the range) during the treatment of the subject. The daily or weekly administration of the first EGFR inhibitor can be at different amounts on different days or during different weeks. For example, the treatment can comprise weekly administration of the first EGFR inhibitor at 400 mg/m² on day 1, 250 mg/m² on day 8, and 250 mg/m² on day 15. For example, the treatment can comprise daily or weekly administration of the first EGFR inhibitor at 0.1 mg to 20 mg during week 1, 0.25 mg to 50 mg during week 2, 0.5 mg to 100 mg during week 3, 1 mg to 200 mg during week 4, and 2 mg to 400 mg during week 5 and beyond. For example, the treatment can comprise daily or weekly administration of the first EGFR inhibitor at 0.1 mg to 100 mg on day 1, 0.2 mg to 200 mg on day 2, 0.4 mg to 400 mg on day 3, and 0.4 mg to 400 mg or 0.6 mg to 600 mg on day 4 and beyond. For example, the first EGFR inhibitor can be administered at a daily or weekly dose of about 0.01 mg, 0.05 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 2 mg, 3 mg, 4 mg, 5 mg, 6 mg, 7 mg, 8 mg, 9 mg, 10 mg, or a number or a range between any two of these values. In some embodiments, the daily or weekly dose of the first EGFR inhibitor can be, or be about, 0.005 mg/m², 0.01 mg/m², 0.05 mg/m², 0.1 mg/m², 0.15 mg/m², 0.2 mg/m², 0.25 mg/m², 0.3 mg/m², 0.35 mg/m², 0.4 mg/m², 0.45 mg/m², 0.5 mg/m², 0.55 mg/m², 0.6 mg/m², 0.65 mg/m², 0.7 mg/m², 0.75 mg/m², 0.8 mg/m², 0.85 mg/m², 0.9 mg/m², 0.95 mg/m², 1 mg/m², 2 mg/m², 3 mg/m², 4 mg/m², 5 mg/m², 6 mg/m², 7 mg/m², 8 mg/m², 9 mg/m², 10 mg/m², or a number or a range between any two of these values. In some embodiments, the daily or weekly dose of the first EGFR inhibitor can be, or be about, 100 mg/m², 110 mg/m², 120 mg/m², 130 mg/m², 140 mg/m², 150 mg/m², 200 mg/m², 250 mg/m², 300 mg/m², 350 mg/m², 400 mg/m², 450 mg/m², 500 mg/m², or a number or a range between any two of these values. In some embodiments, the daily or weekly dose of the first EGFR inhibitor can be, or be about, 0.005 mg/kg, 0.01 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, or a number or a range between any two of these values. In some embodiments, the daily or weekly dose of the first EGFR inhibitor can be, or be about, 100 mg/kg, 110 mg/kg, 120 mg/kg, 130 mg/kg, 140 mg/kg, 150 mg/kg, 200 mg/kg, 250 mg/kg, 300 mg/kg, 350 mg/kg, 400 mg/kg, 450 mg/kg, 500 mg/kg, or a number or a range between any two of these values. In some embodiments, a patient is administered an effective dose of a corticosteroids (e.g., dexamethasone), a diphenhydramine, and/or H2 antagonists (e.g., cimetidine or famotidine) prior to administering the first EGFR inhibitor.

In some embodiments, the first EGFR inhibitor is an antibody (e.g., cetuximab). A maximum concentration (Cₘₐₓ) of the first EGFR inhibitor in a blood of the subject (during the treatment or after the treatment) when the first EGFR inhibitor is administered alone or in combination with the PLK1 inhibitor can be from about 10 to about 200 μg/mL (microgram per mL). For example, the Cₘₐₓ of the first EGFR inhibitor in a blood of the subject when the first EGFR inhibitor is administered alone or in combination with the PLK1 inhibitor can be, or be about, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 120 μg/mL, 130 μg/mL, 140 μg/mL, 150 μg/mL, 160 μg/mL, 170 μg/mL, 180 μg/mL, 190 μg/mL, 195 μg/mL, 200 μg/mL, or a number or a range between any two of these values, or any value between 1 μg/mL to 200 μg/mL.

The treatment of the present disclosure can comprise administration of a PLK1 inhibitor (e.g., onvansertib) for a desired duration in one or more cycles. In some embodiments, the PLK1 inhibitor (e.g., onvansertib) is administered for 1 to 10 cycles, for example, 1 to 9 cycles, 1 to 8 cycles, 1 to 7 cycles, 1 to 6 cycles, 1 to 5 cycles, 1 to 4 cycles, 1 to 3 cycles, 1 to 2 cycles, or 1 cycle. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more.

In some embodiments, the administration of the PLK1 inhibitor can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be once, twice, three times, four times, or more on a day when the PLK1 inhibitor is administered to the patient. The administration can be, for example, once every two days, every three days, every four days, every five days, every six days, or every seven days. The length of the desired duration can vary, for example, 1, 2, 3, 4, 5, 6, 7, 8, 9, 10, 11, 12, 13, 14, 15, 16, 17, 18, 19, 20, 21, 22, 23, 24, 25, 26, 27, 28, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of e.g., the PLK1 inhibitor for four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). In some embodiments, the treatment can comprise administration of the PLK1 inhibitor (e.g., onvansertib) for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the e.g., the PLK1 inhibitor in a single cycle of the treatment can be continuous or with one or more intervals (e.g., one day or two days of break). In some embodiments, the treatment comprises administration of the PLK1 inhibitor (e.g., onvansertib) for five days in a cycle of 14 to 28 days. In some embodiments, the PLK1 inhibitor (e.g., onvansertib) is administered daily for about 14 days, followed by a 7-day off. In some embodiments, the PLK1 inhibitor (e.g., onvansertib) is administered orally. In some embodiments the PLK1 inhibitor (e.g., onvansertib) is administered without any catch-up doses.

The PLK1 inhibitor (e.g., onvansertib) can be administered to the subject in need thereof on twenty days (e.g., Days 1-10 and 15-24) during a 28-day cycle. The twenty days can be, for example, a continuous daily administration for ten days (e.g., Days 1-10) and another continuous daily administration (e.g., Days 15-24) for ten days, or a continuous daily administration for four sets of five days (e.g., Days 1-5, 8-12, 15-19, and 22-26). In some embodiments, the PLK1 inhibitor (e.g., onvansertib) is administered to the subject in need thereof on twenty-one days (e.g., Days 1-21) during a 28-day cycle. In some embodiments, for example when the patient is identified to have low tolerance to the PLK1 inhibitor (e.g., onvansertib), the PLK1 inhibitor is administered to the subject in need thereof on ten days (e.g., Days 1-5 and 15-19) during a 28-day cycle. The ten days can be, for example, a continuous daily administration for ten days (e.g., Days 1-10) or two continuous daily admiration for five days each (e.g., Days 1-5 and Days 15-19). In some embodiments, the PLK1 inhibitor (e.g., onvansertib) is administered to the subject in need thereof daily throughout the whole cycle (e.g., daily for 28 days in a cycle of 28 days). Depending on the needs of inhibition/reversion of cancer progression in the subject, the subject can receive one, two, three, four, five, six, or more cycles of treatment. For combination treatment, the administration cycles, dosing schedules, and/or dosage amounts of the first EGFR inhibitor and the PLK1 inhibitor can be the same or different. For combination treatment, the administration cycle, dosing schedule, and/or dosage amount of the first EGFR inhibitor can be adjusted according to the administration cycle, dosing schedule, and/or dosage amount of the PLK1 inhibitor. For example, the first EGFR inhibitor can be administered three times in a 28-day cycles (e.g., daily dose on Days 1, 8 and 15), which corresponds to a 28-day cycle for administration of the PLK1 inhibitor (e.g., onvansertib).

The treatment can comprise administration of the PLK1 inhibitor (e.g., onvansertib) at, or at about, 6 mg/m²-90 mg/m² drug/body surface area, for example, as a daily dose. For example, the treatment can comprise daily administration of the PLK1 inhibitor (e.g., onvansertib) at, or at about, 6 mg/m², 8 mg/m², 10 mg/m², 12 mg/m², 14 mg/m², 16 mg/m², 18 mg/m², 20 mg/m², 23 mg/m², 27 mg/m², 30 mg/m², 35 mg/m², 40 mg/m², 45 mg/m², 50 mg/m², 55 mg/m², 60 mg/m², 65 mg/m², 70 mg/m², 80 mg/m², 85 mg/m², 90 mg/m², a number or a range between any two of these values, or any value between 8 mg/m²-90 mg/m². In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the PLK1 inhibitor (e.g., onvansertib) is administered at 12 mg/m² on twenty days (e.g., Days 1-10 and 15-24) during a 28-day cycle. In some embodiments, the PLK1 inhibitor (e.g., onvansertib) is administered at 15 mg/m² on ten days (e.g., Days 1-5 and 15-19) during a 28-day cycle. In some embodiments, the PLK1 inhibitor (e.g., onvansertib) is administered at 8 mg/m² or 10 mg/m² everyday (e.g., Days 11-28) during a 28-day cycle. In some embodiments, the PLK1 inhibitor (e.g., onvansertib) is administered at 45 mg/m² 5 days a week during a 18-day cycle. In some embodiments, the PLK1 inhibitor (e.g., onvansertib) is administered at 45 mg/m² 5 days a week during a 32-day cycle. In some embodiments, the PLK1 inhibitor (e.g., onvansertib) is administered at 45 mg/m² 5 days a week during a 39-day cycle. In some embodiments, the PLK1 inhibitor (e.g., onvansertib) is administered at 45 mg/m² 5 days a week during a 45-day cycle. In some embodiments, the PLK1 inhibitor (e.g., onvansertib) is administered at 45 mg/m² 5 days a week during a cycle (e.g., 30-day, 31-day, 32-day, 33-day, 34-day, 35-day, 36-day, 37-day, 38-day, 39-day or 40-day cycle) with no administration of the PLK1 inhibitor (e.g., onvansertib) for one week.

The treatment can comprise administration of the PLK1 inhibitor (e.g., onvansertib) at, or at about, 5 mg-200 mg, for example, as a daily dose. For example, the treatment can comprise daily administration of the PLK1 inhibitor (e.g., onvansertib) at, or at about, 5 mg, 6 mg, 8 mg, 10 mg, 12 mg, 14 mg, 16 mg, 18 mg, 20 mg, 23 mg, 27 mg, 30 mg, 35 mg, 40 mg, 45 mg, 50 mg, 55 mg, 60 mg, 65 mg, 70 mg, 80 mg, 85 mg, 90 mg, a number or a range between any two of these values, or any value between 5 mg-200 mg. In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the PLK1 inhibitor (e.g., onvansertib) is administered at 12 mg on twenty days (e.g., Days 1-10 and 15-24) during a 28-day cycle. In some embodiments, the PLK1 inhibitor (e.g., onvansertib) is administered at 15 mg on ten days (e.g., Days 1-5 and 15-19) during a 28-day cycle. In some embodiments, the PLK1 inhibitor (e.g., onvansertib) is administered at 8 mg or 10 mg everyday (e.g., Days 11-28) during a 28-day cycle. In some embodiments, the PLK1 inhibitor (e.g., onvansertib) is administered at 45 mg 5 days a week during an 18-day cycle. In some embodiments, the PLK1 inhibitor (e.g., onvansertib) is administered at 45 mg 5 days a week during a 32-day cycle. In some embodiments, the PLK1 inhibitor (e.g., onvansertib) is administered at 45 mg 5 days a week during a 39-day cycle. In some embodiments, the PLK1 inhibitor (e.g., onvansertib) is administered at 45 mg 5 days a week during a 45-day cycle. In some embodiments, the PLK1 inhibitor (e.g., onvansertib) is administered at 45 mg 5 days a week during a cycle (e.g., 30-day, 31-day, 32-day, 33-day, 34-day, 35-day, 36-day, 37-day, 38-day, 39-day or 40-day cycle) with no administration of the PLK1 inhibitor (e.g., onvansertib) for one week. Besides body surface area based dosing and flat-fixed dosing, other dosing unit (mg/kg) can be used. For example, the treatment can comprise administration of the PLK1 inhibitor (e.g., onvansertib) at, or at about, 0.05 mg/kg-50 mg/kg, for example, as a daily dose. In some embodiments, the PLK1 inhibitor (e.g., onvansertib) is administered at 45 mg/kg 5 days a week during a 18-day cycle. In some embodiments, the PLK1 inhibitor (e.g., onvansertib) is administered at 45 mg/kg 5 days a week during a 32-day cycle. In some embodiments, the PLK1 inhibitor (e.g., onvansertib) is administered at 45 mg/kg 5 days a week during a 39-day cycle. In some embodiments, the PLK1 inhibitor (e.g., onvansertib) is administered at 45 mg/kg 5 days a week during a 45-day cycle. In some embodiments, the PLK1 inhibitor (e.g., onvansertib) is administered at 45 mg/kg 5 days a week during a cycle (e.g., 30-day, 31-day, 32-day, 33-day, 34-day, 35-day, 36-day, 37-day, 38-day, 39-day or 40-day cycle) with no administration of the PLK1 inhibitor (e.g., onvansertib) for one week.

In some embodiments, the daily dose of the PLK1 inhibitor can be adjusted (e.g., increased or decreased with the range) during the treatment, or during a single cycle (e.g., the first cycle, the second cycle, the third cycle, and a subsequent cycle) of the treatment, for the subject.

A maximum concentration (Cₘₐₓ) of the PLK1 inhibitor (e.g., onvansertib) in a blood of the subject (during the treatment or after the treatment) when the PLK1 inhibitor is administered alone or in combination with the first EGFR inhibitor (e.g., cetuximab) can be from about 100 nmol/L to about 1500 nmol/L. For example, the Cₘₐₓ of the PLK1 inhibitor (e.g., onvansertib) in a blood of the subject when the PLK1 inhibitor is administered alone or in combination with the first EGFR inhibitor can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the first EGFR inhibitor can be from about 1000 nmol/L·hour to about 400000 nmol/L·hour. For example, the AUC of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the first EGFR inhibitor can be, or be about, 1000 nmol/L·hour, 5000 nmol/L·hour, 10000 nmol/L·hour, 15000 nmol/L·hour, 20000 nmol/L·hour, 25000 nmol/L·hour, 30000 nmol/L·hour, 35000 nmol/L·hour, 40000 nmol/L·hour, a range between any two of these values, or any value between 1000 nmol/L·hour and 400000 nmol/L·hour.

A time (Tₘₐₓ) to reach a maximum concentration of the PLK1 inhibitor (e.g., onvansertib) in a blood of the subject when the PLK1 inhibitor is administered alone or in combination with the first EGFR inhibitor can be from about 1 hour to about 5 hours. For example, the time (Tₘₐₓ) to reach a maximum concentration of the PLK1 inhibitor (e.g., onvansertib) in a blood of the subject when the PLK1 inhibitor is administered alone or in combination with the first EGFR inhibitor can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with the EGFR inhibitor (e.g., the first EGFR inhibitor) can be from about 10 hours to about 60 hours. For example, the elimination half-life (T_{1/2}) of the PLK1 inhibitor (e.g., onvansertib) in a blood of the subject when the PLK1 inhibitor is administered alone or in combination with the first EGFR inhibitor can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

Patients administered one or more dose cycles of the PLK1 inhibitor or one or more dose cycles of the PLK1 inhibitor in combination with one or more cycles of the first EGFR inhibitor can exhibit very tolerable AE, including in some cases undetectable definite AE or definite SAE. A remarkable, but unlikely result is the finding that the patient has no probable or even possible AE or SAE. In some embodiments, treated with the PLK1 inhibitor alone or the combined therapy of the first EGFR inhibitor and PLK1 inhibitor can lead to remarkable therapeutic effect. A therapeutic effect greater than the therapeutic effect predicted from in vitro or in silico is indicative of a surprising result. A therapeutic dose lower than the therapeutic dose predicted from in vitro or in silico is indicative of a surprising result. It is expected that the disclosed method of treatment can mitigate disease progression in patients. A highly positive result is the finding that the therapy of the disclosure can lead to stable disease. A remarkable, but unlikely result is the finding of a complete response or complete remission of the cancer, a progression-free survival, an overall survival rate exceeding values predicted from in vitro or in silico analysis, is free of any measurable lesion, free of any target lesion, or free of any malignant lymph nodes.

The first and/or second EGFR inhibitor can be erlotinib, lapatinib, AZD8931, WZ4002, panitumumab, vandetanib, icotinib, afatinib, brigatinib, CO-1688, AZD-4769, poziotinib, CUDC-101, S-222611, AC-480, imgatuzumab, sapitinib, TAS-2913, theiiatinib, XGFR-2421, HM-61713B, epitinib, NRC-2694, MLBS-42, JRP-890, cetuximab, AL-6802, TAK-285, BGB-102, AEE788, gefitinib, DMS-3008, TX-2036, KI-6783, KI-6896; or a pharmaceutically acceptable salt, hydrate, solvate or prodrug thereof; or any combination thereof. The first and/or second EGFR inhibitor can be cetuximab or panitumumab. The first and second EGFR inhibitors can be the same. The first and second EGFR inhibitors can be different.

The PLK1 inhibitor can be onvansertib (NMS-P937), BI2536, volasertib (BI 6727), GSK461364, adavosertib (AZD1775), CYC140, HMN-176, HMN-214, rigosertib (ON-01910), MLN0905, TKM-080301, TAK-960, GTPL10072, Ro3280; or a pharmaceutically acceptable salt, hydrate, solvate or prodrug thereof; or any combination thereof. The PLK1 inhibitor can be onvansertib.

### Additional Cancer Therapeutics or Therapy

Methods, compositions and kits disclosed herein can be used for treating cancer, for example RAS^{WT} colorectal cancer. In some embodiments, a method for treating cancer comprises administrating a PLK-1 inhibitor (e.g., onvansertib) or a combination therapy comprising PLK-1 inhibitor (e.g., onvansertib) and an EGFR inhibitor to a subject (e.g., a patient) in need thereof. The method can comprise administering a therapeutically effective amount of the PLK-1 inhibitor or both the PLK-1 inhibitor and the EGFR inhibitor (e.g., the first EGFR inhibitor) to a subject in need thereof. The treatment can comprise administration of at least one additional cancer therapeutics or cancer therapy. The treatment can comprise administration a therapeutically effective amount of at least one additional cancer therapeutics or cancer therapy. The PLK-1 inhibitor and/or the first EGFR inhibitor and the cancer therapeutics or cancer therapy can, for example, co-administered simultaneously or sequentially.

In some embodiments, the method does not comprise administration of additional cancer therapeutics or therapies for the cancer. In some embodiments, the method comprises administration of the PLK-1 inhibitor alone (e.g., without the administration of an additional therapy for treating cancer). For example, in some embodiments, the method does not comprise administering irinotecan, bevacizumab, or both to the subject with the RAS^{WT} colorectal cancer. In some embodiments, the method comprises administration of the PLK-1 inhibitor and the first EGFR inhibitor, and does not comprise administration of any additional therapy for treating cancer. In some embodiments, the method comprises administering a PLK1 inhibitor and a first EGFR inhibitor to a subject with the RAS^{WT} colorectal cancer, thereby inhibiting or reducing progression of the RAS^{WT} colorectal cancer in the subject and does not comprise administering irinotecan, bevacizumab, or both to the subject with the RAS^{WT} colorectal cancer. In some embodiments, the subject received a prior therapy for cancer. In some embodiments, the subject did not respond to or developed resistance to a prior therapy for cancer. In some embodiments, the prior therapy comprises chemotherapy, radiation therapy, immunotherapy, hormone therapy, hyperthermia, photodynamic therapy, stem cell therapy, surgery, targeted therapy, or any combination thereof. The prior therapeutics or therapies for cancer can comprise chemotherapy, radiation therapy, immunotherapy, hormone therapy, hyperthermia, photodynamic therapy, stem cell therapy, surgery, targeted therapy, or any combination thereof.

The additional therapeutics or therapies for cancer can comprise chemotherapy, radiation therapy, immunotherapy, hormone therapy, hyperthermia, photodynamic therapy, stem cell therapy, surgery, targeted therapy, or any combination thereof.

Exemplary chemotherapeutic agents are described below. The chemotherapeutic agent can be an alkylating agent (including nitrosoureas). Alkylating agents damage cell DNA to prevent cancer cells from dividing. Nitrosoureas are a particular type of alkylating agent. Unlike other alkylating agents, nitrosoureas can travel into the brain and kill cancer cells there. Nitrosoureas are used to treat some brain tumors. Exemplary alkylating agents include: Altretamine, Bendamustine, Busulfan, Carboplatin, Chlorambucil, Cisplatin, Cyclophosphamide, Dacarbazine, Ifosfamide, Mechlorethamine, Melphalan, Oxaliplatin, Procarbazine, Temozolomide, Thiotepa, and Trabectedin. Exemplary nitrosoureas include: Carmustine, Lomustine, and Streptozocin.

The chemotherapeutic agent can be an antimetabolite. Antimetabolites prevent cancer cells from making the genetic material they need to create new cells. Exemplary antimetabolites include: 5-fluorouracil, 6-mercaptopurine, Azacitidine, Capecitabine, Cladribine, Clofarabine, Cytarabine, Decitabine, Floxuridine, Fludarabine, Gemcitabine, Hydroxyurea, Methotrexate, Nelarabine, Pemetrexed, Pentostatin, Pralatrexate, Thioguanine, and Trifluridine/tipiracil combination.

The chemotherapeutic agent can be a topoisomerase inhibitor. Topoisomerase inhibitors inhibit DNA replication. Stopping this enzyme prevents cancer cells from multiplying and can also damage the cell DNA. Exemplary topoisomerase inhibitors include: Etoposide, Irinotecan, Irinotecan liposomal, Mitoxantrone (also classified as an antitumor antibiotic, see below), Teniposide, and Topotecan.

The chemotherapeutic agent can be a mitotic inhibitor (e.g., plant alkaloids). Mitotic inhibitors are also called plant alkaloids because they are made of the same material plants use to protect against predators. Exemplary mitotic inhibitors include: Cabazitaxel, Docetaxel, Nab-paclitaxel, Paclitaxel, Vinblastine, Vincristine, Vincristine liposomal, and Vinorelbine.

The chemotherapeutic agent can be an antitumor antibiotic (including anthracyclines). Antitumor antibiotics prevent the cancer-cell DNA from replication. Sometimes, they induce DNA damage. Anthracyclines are a specific type of antitumor antibiotic. Exemplary anthracyclines include: Daunorubicin, Doxorubicin, Doxorubicin liposomal, Epirubicin, Idarubicin, Mitoxantrone, and Valrubicin. Other antitumor antibiotics include Bleomycin, Dactinomycin, and Mitomycin-C.

Additional exemplary chemotherapeutic agents include, but are not limited to: All-trans-retinoic acid, Arsenic trioxide, Asparaginase, Eribulin, Ixabepilone, Mitotane, Omacetaxine, Pegaspargase, Procarbazine, Romidepsin, and Vorinostat. In some embodiments, the chemotherapeutic agent is a combination therapy comprising folinic acid, 5-fluorouracil, and irinotecan (e.g., FOLFIRI). In some embodiments, the chemotherapeutic agent is a combination therapy comprising Leucovorin (e.g., folinic acid), 5-fluorouracil, and oxaliplatin (e.g., FOLFOX).

In some embodiments, the immunotherapy comprises T-cell therapy (e.g., CAR-T), antibodies (e.g., monoclonal antibodies), vaccines, and/or immune modulators. In some embodiments, the antibody can be rituximab (Rituxan®), alemtuzumab (Campath®), Bevacizumab (Avastin®), Cetuximab (Erbitux®), panitumumab (Vectibix®), and trastuzumab (Herceptin®), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, 1311-tosit.umomab, ado-trastuzumab emtansine, lapatinib (Tykerb®), pertuzumab (Perjeta™), ado-trastuzumab emtansine (Adcyla™), regorafenib (Stivarga®), sunitinib (Sutent®), Denosumab (Xgeva®), sorafenib (Nexavar®), pazopanib (Votrient®), axitinib (Inita®), dasatinib (Sprycel®), nilotinib (Tasigna®), bosutinib (Bosulif®), ofatumumab (Arzerra®), obinutuzumab (Gazyva™), ibrutinib (Imbruvica™), idelalisib (Zydelig®), crizotinib (Xalkori®), erlotinib (Tarceva®), afatimb dimaleate (Giiotrif®), ceritinib (LDK378/Zykadia), Tositumomab and 1311-tositumomab (Bexxar®), ibritumomab tiuxetan (Zevalin®), brentuximab vedotin (Adcetris®), bortezomib (Velcade®), siltuximab (Sylvant™), trametinib (Mekinist®), dabrafenib (Tafmlar®), pembrolizimiab (Keytruda®), carfilzomib (Kyprolis®), Ramucirumab (Cyramza™), Cabozantinib (Cometriq™), or vandetanib (Caprelsa®).

In some embodiments, the immunotherapy can be a cytokine such as interferons (INFs), interleukins (ILs), or hematopoietic growth factors. The therapeutic agent can be INF-a, IL-2, Aldesleukin, IL-2, Erythropoietin, Granulocyte-macrophage colony-stimulating factor (GM-CSF) or granulocyte colony-stimulating factor. In some embodiments, the immunotherapy comprises an immunomodulator such as (Thalomid®), (Revlimid®), pomalidomide (Pomalyst®), and/or imiquimod (Aldara®, Zyclara®).

In some embodiments, the additional treatment or therapy can be a targeted therapy such as toremifene (Fareston®), fulvestrant (Faslodex®), anastrozole (Arimidex®), exemestane (Aromasin®), letrozole (Femara®), ziv- aflibercept (Zaltrap®), alitretinoin (Panretin®), temsirolimus (Torisel®), Tretinoin (Vesanoid®), denileukin diftitox (Ontak®), vorinostat (Zoiinza®), romidepsin (Istodax®), bexarotene (Targretin®), pralatrexate (Foiotyn®), lenaliomide (Revlimid®), belinostat (Beleodaq™), lenaliomide (Revlimid®), pomalidomide (Pomalyst®), Cabazitaxel (Jevtana®), enzaluiamide (Xtandi®), abiraterone acetate (Zytiga®), radium 223 chloride (Xofigo®), or everolimus (Afmitor®). Additionally, the therapeutic agent can be an epigenetic targeted drug such as HDAC inhibitors, kinase inhibitors, DNA methyltransferase inhibitors, histone demethylase inhibitors, or histone methylation inhibitors. The epigenetic drugs can be Azacitidine (Vidaza), Decitabine (Dacogen), Vorinostat (Zoiinza), Romidepsin (Istodax), or Ruxolitinib (Jakafi).

### Methods for Predicting/Determining Treatment Efficacy and Status of Cancer

Also disclosed herein include methods, compositions, kits, and systems for predicting/determining clinical outcome for a combination treatment of cancer of the present disclosure, monitoring of the combination treatment, predicting/determining responsiveness of a subject to the combination treatment, determining the status of the cancer in a subject, and improving combination treatment outcome. The methods, compositions, kits and systems can be used to guide the combination treatment, provide combination treatment recommendations, reduce or avoid unnecessary ineffective combination treatment for patients. ctDNA can be analyzed to predict/determine clinical outcome for cancer treatment using a PLK-1 inhibitor or a combination of a PLK-1 inhibitor and an EGFR inhibitor of the present disclosure, monitor the combination treatment, predict/determine responsiveness of a subject to the combination treatment, determine cancer status in a subject, improve combination treatment outcome, guide combination treatment, provide combination treatment recommendations, and/or to reduce or avoid ineffective combination treatment. ctDNA can be analyzed to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or to reduce or avoid ineffective cancer treatment. Such analysis of ctDNA has been described in PCT Application No. PCT/US2021/013287, the content of which is incorporated herein by reference in its entirety.

A method of determining responsiveness of a subject to a combination treatment comprising a PLK-1 inhibitor or a PLK-1 inhibitor and an EGFR inhibitor of the disclosure can comprise, for example, analyzing circulating tumor DNA (ctDNA) of a subject with cancer, the subject is undergoing a treatment and/or has received the combination treatment, thereby determining the responsiveness of the subject to the combination treatment. In some embodiments, determining the responsiveness of the subject comprises determining if the subject is a responder of the treatment, if the subject is or is going to be in CR, or if the subject is or is going to be in partial remission (PR). For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the cancer treatment.

In some embodiments, the first time point is prior to or immediately prior to the combination treatment, and at least one of the one or more additional time points are at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment.

In some embodiments, the first cycle of the combination treatment is immediately prior to the second cycle of the combination treatment. In some embodiments, the method comprises continuing the combination treatment to the subject if the subject is indicated as responsive to the combination treatment. In some embodiments, the method comprises discontinuing the combination treatment to the subject and/or starting a different combination treatment to the subject if the subject is not indicated as responsive to the combination treatment.

Disclosed herein include methods of determining cancer status of a subject, comprising analyzing circulating tumor DNA (ctDNA) of a subject, thereby determining cancer status of the subject. The subject can be a subject undergoing a current combination treatment comprising a PLK-1 inhibitor or a PLK-1 inhibitor and an EGFR inhibitor of the present disclosure, a subject that has received a prior combination treatment of the present disclosure, and/or a subject that is in remission for the cancer. The subject in remission for cancer can be in complete remission (CR), or in partial remission (PR).

In some embodiments, analyzing the ctDNA comprises detecting variant allele frequency in the ctDNA. In some embodiments, analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, an increase in the variant allele frequency at the additional sample(s) relative to the first sample indicates that the subject is at risk of cancer relapse or is in cancer relapse.

In some embodiments, the first time point is prior or immediately prior to the combination treatment, and the one or more additional time points are at the end of or after at least a cycle of the combination treatment, optionally the cycle of the combination treatment is the first cycle of the combination treatment. In some embodiments, the first time point is prior or immediately prior to a first cycle of the combination treatment, and the one or more additional time points are at the end of or after a second cycle of the combination treatment, optionally the first cycle of the combination treatment is immediately prior to the second cycle of the combination treatment.

In some embodiments, the method comprises starting an additional treatment to the subject if the subject is indicated as in cancer relapse. The additional treatment can be the same or different from the current or prior combination treatment.

The variant allele frequency in ctDNA can be determined, for example, by total mutation count in the ctDNA in each of the first sample and one or more additional samples, or by the mean variant allele frequency in each of the first sample and one or more additional samples. In some embodiments, the variant allele frequency is mutant allelic frequency (MAF) for a driver mutation of the cancer (e.g., ovarian cancer, breast cancer, prostate cancer, colorectal cancer, pancreatic cancer, or a combination thereof). In some embodiments, the variant allele frequency is MAF for one or more driver mutations of the cancer (e.g., ovarian cancer, breast cancer, prostate cancer, colorectal cancer, pancreatic cancer, or a combination thereof). In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is, or is about, 0.01 to −0.10. In some embodiments, the MAF threshold is, or is about, 0.06. In some embodiments, the MAF threshold is, or is about, 0.05.

In some embodiments, the first sample comprises ctDNA from the subject before treatment, and the one of additional samples comprises ctDNA from the subject after treatment. In some embodiments, the driver mutation is a mutation in one of the below 75 genes: ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCH1, NPM1, NRAS, PDGFRA, PHF6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2. In some embodiments, at least one of the one or more the driver mutations is a mutation in in the 75 genes. In some embodiments, one or more the driver mutations are mutations in the 75 genes.

The driver mutation or at least one of the one or more driver mutations can be in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1. In some embodiments, the method further comprises determining variant allele frequency in one or more of the ctDNA, PBMCs and BMMCs of the subject.

The ctDNA can be analyzed using, for example, polymerase chain reaction (PCR), next generation sequencing (NGS), and/or droplet digital PCR (ddPCR). The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

In some embodiments, the method comprises analyzing ctDNA of the subject before the treatment. In some embodiments, the treatment comprises one or more cycles, and the ctDNA is analyzed before, during and after each cycle of the treatment. Each cycle of treatment can be at least 21 days. In some embodiments, each cycle of treatment is from about 21 days to about 28 days. In some embodiments, the subject is human.

Disclosed herein include methods of improving treatment outcome for the cancer. The method can comprise: detecting variant allele frequency in circulating tumor DNA (ctDNA) obtained from a subject at a first time point in a first sample before the subject undergoes a combination treatment comprising a PLK-1 inhibitor or a PLK-1 inhibitor and an EGFR inhibitor of the present disclosure; detecting variant allele frequency in ctDNA obtained from the subject at one or more additional time points in one or more additional samples after the subject undergoes the combination treatment; determining the difference of the variant allele frequency in ctDNA between the first and at least one of the one or more additional samples, a decrease in the variant allele frequency in at least one of the additional samples relative to the first sample indicates the subject as responsive to the combination treatment; and continuing the combination treatment to the subject if the subject is indicated as responsive to the combination treatment, or discontinuing the combination treatment to the subject and/or starting a different cancer treatment to the subject if the subject is not indicated as responsive to the combination treatment.

Also disclosed herein include methods of treating cancer (e.g., RAS^{WT} colorectal cancer). The method can comprise: administering a combination treatment comprising a PLK-1 inhibitor or a PLK-1 inhibitor and an EGFR inhibitor of the present disclosure to a subject in need thereof; determining a decrease, relative to a variant allele frequency in a first sample of the subject obtained at a first time point before the subject receives the combination treatment, in a variant allele frequency in a second sample of the subject obtained at a second time point after the subject receives the combination treatment; and continuing with the combination treatment. In some embodiments, the subject is a subject newly diagnosed with cancer, for example a subject that has not received any prior cancer treatment before the combination treatment. In some embodiments, the subject has received prior cancer treatment and was in remission for the cancer, for example a subject in complete remission (CR), or in partial remission (PR) after receiving the prior combination treatment.

The first time point can be, for example, prior or immediately prior to the combination treatment. The at least one of the one or more additional time points can be, for example, at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment. In some embodiments, the first cycle of the combination treatment is immediately prior to the second cycle of the combination treatment.

The variant allele frequency in ctDNA can be determined, for example, by total mutation count in the ctDNA in each of the first sample and one or more additional samples, and/or by the mean variant allele frequency in each of the first sample and one or more additional samples. In some embodiments, the variant allele frequency is mutant allelic frequency (MAF) for a driver mutation of the cancer (e.g., ovarian cancer, breast cancer, prostate cancer, colorectal cancer, pancreatic cancer, or a combination thereof). In some embodiments, the variant allele frequency is mutant allelic frequency (MAF) for one or more driver mutations of the cancer (e.g., ovarian cancer, breast cancer, prostate cancer, colorectal cancer, pancreatic cancer, or a combination thereof). In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is-0.05.

The driver mutation can be at least one of the following 75 genes: ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCH1, NPM1, NRAS, PDGFRA, PHF6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2. The one or more driver mutations are mutations in the 75 genes. In some embodiments, the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1.

In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1.

In some embodiments, the method further comprises determining variant allele frequency in one or more of the ctDNA, PBMCs and BMMCs of the subject. The variant allele frequency in ctDNA can be detected, for example, using polymerase chain reaction (PCR) or next generation sequencing (NGS). In some embodiments, the variant allele frequency in ctDNA is detected using droplet digital PCR (ddPCR).

At least one of the first sample, the one or more additional samples, and the second sample can be derived from whole blood of the subject, plasma of the subject, serum of the subject, or a combination thereof. In some embodiments, the ctDNA is from whole blood of the subject, plasma of the subject, serum of the subject, or a combination thereof.

In some embodiments, the subject whose ctDNA is analyzed is undergoing or will be undergoing treatment for the cancer. The method can comprise analyzing ctDNA of the subject before the treatment. The treatment can comprise one or more cycles, and the ctDNA is analyzed before, during and after one or more cycles of the treatment. For example, the ctDNA can be analyzed before, during and after two or more cycle of the treatment, three or more cycle of the treatment, or each cycle of the treatment. Each cycle of treatment can be at least 21 days, for example, 21 days, 22 days, 23 days, 24 days, 25 days, 26 days, 27 days, 28 days, 29 days, 30 days, or more, or a range between any two of these values. In some embodiments, each cycle of treatment is from about 21 days to about 28 days. In some embodiments, each cycle of treatment is from 21 days to 28 days. In some embodiments, the subject is human.

In some embodiments, the subject has received at least one prior cancer treatment. In some embodiments, the prior treatment does not comprise the use of a PLK1 inhibitor. The PLK1 inhibitor can be onvansertib. In some embodiments, the subject was in remission for cancer. In some embodiments, the subject in remission for cancer was in complete remission (CR) or in partial remission (PR). The method can comprise determining cancer status of the subject. The method can comprise determining responsiveness of the subject to the treatment with the PLK1 inhibitor, or treatment with the PLK1 inhibitor and the first EGFR inhibitor. The subject can be human. In some embodiments, the subject achieves a complete response.

In some embodiments, the volume of one or more tumors in the subject increases by at most 25% (e.g., at most 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or a number or a range between any two of these values) relative to the volume of the one or more tumors prior to the administering, following at least one cycle of treatment. In some embodiments, the volume of one or more tumors in the subject decreases by at least 25% (e.g., at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, or a number or a range between any two of these values or more) relative to the volume of the one or more tumors prior to the administering, following at least one cycle of treatment. In some embodiments, the increase or decrease is relative to the volume prior to administration of the PLK-1 inhibitor or the PLK-1 inhibitor and the EGFR inhibitor.

### Methods and Compositions for Sensitizing Cancer Cells to an EGFR Inhibitor

Disclosed herein include methods of sensitizing cancer cells to an EGFR inhibitor. In some embodiments, the method comprises: contacting cancer cells with a composition comprising onvansertib, thereby sensitizing the cancer cells to the EGFR inhibitor.

The cancer cells can be RAS^{WT} colorectal cancer cells. The EGFR inhibitor can be cetuximab or panitumumab. In some embodiments, contacting cancer cells with the composition occurs in vitro, ex vivo, and/or in vivo.

In some embodiments, contacting cancer cells with the composition is in a subject. In some embodiments, the subject did not respond to, or is known to be resistant to, the EGFR inhibitor. In some embodiments, the subject had prior treatment with the EGFR inhibitor. The subject can be a mammal. The mammal can be a human.

The method can comprise determining sensitization of the cancer cells to the EGFR inhibitor after being contacted with the composition. The method can comprise contacting the cancer cells with the EGFR inhibitor. In some embodiments, contacting the cancer cells with the EGFR inhibitor occurs in the subject. The method can comprise determining the response of the subject to the EGFR inhibitor.

In some embodiments, the volume of one or more tumors comprising the cancer cells in the subject increases by at most 25% (e.g., at most 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or a number or a range between any two of these values) relative to the volume of the one or more tumors prior to the contacting. In some embodiments, the volume of one or more tumors comprising the cancer cells in the subject decreases by at least 25% (e.g., at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 200%, or a number or a range between any two of these values or more) relative to the volume of the one or more tumors prior to the contacting. In some embodiments, the increase or decrease is relative to the volume prior to the contacting.

Contacting the cancer cells with the EGFR inhibitor can be concurrent with the contacting the cancer cells with the composition, or after the contacting the cancer cells with the composition (e.g., the composition comprising onvansertib). For example, contacting the cancer cells with the EGFR inhibitor can be 1 h to 24 h (e.g., 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, or a number or a range between any two of these values or more) after the contacting the cancer cells with the composition (e.g., the composition comprising onvansertib). The cancer cells can be cells of EGFR-amplified cancer.

The sensitization of the cancer cells can, for example, reduce the colony forming capacity of the cancer cells by, by at least, or by at least about, 20%, 25%, 30%, 35%, 40%, 45%, 50%, 55%, 60%, 65%, 70%, 75%, 80%, 85%, 90%, 95%, or a range between any two of these values. The decrease of the colony-forming capacity of the cancer cells is, in some embodiments, relative to the cancer cells not treated with the composition. The sensitization of the cancer cells can increase the relative number of cells in G2 and/or mitotic stages by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the expression of mitotic markers, such as phosphorylated nucleophosmin (NPM) on Threonine 199, in the cancer cells by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the percentage of cancer cells expressing phosphorylated histone H3 (pHH3) by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the percentage of cancer cells expressing cleaved caspase-3 by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the expression of the apoptotic markers, such as cleaved caspase-3 and cleaved PARP, by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the expression of DNA damage markers, such as γ-H2AX, by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the percentage of cancer cells expressing γ-H2AX by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The increase of the relative number of cells in G2 and/or mitotic stages, of the expression of phosphorylated NPM (Thr199), cleaved caspase 3, cleaved PARP and yH2AX, of the percentage of cancer cells expressing pHH3, cleaved caspase-3 and γ-H2AX is, in some embodiments, relative to those in the cancer cells or subjects not treated with the composition.

### Compositions and Kits

Disclosed herein include kits. In some embodiments, the kit comprises: a PLK1 inhibitor. In some embodiments, the kit comprises: a first EGFR inhibitor. The kit can comprise a manual providing instructions for administering the PLK1 inhibitor or co-administering the PLK1 inhibitor and the first EGFR inhibitor to a subject in need thereof for treating RAS^{WT} colorectal cancer.

The instructions can comprise instructions for co-administrating the PLK1 inhibitor and the first EGFR inhibitor simultaneously. The instructions can comprise instructions for co-administrating the PLK1 inhibitor and the first EGFR inhibitor sequentially.

The instructions can comprise instructions for administering to a subject that did not respond to treatment with a second EGFR inhibitor alone. The instructions can comprise instructions for administering to a subject resistant to the second EGFR inhibitor.

The first and/or second EGFR inhibitor can be erlotinib, lapatinib, AZD8931, WZ4002, panitumumab, vandetanib, icotinib, afatinib, brigatinib, CO-1688, AZD-4769, poziotinib, CUDC-101, S-222611, AC-480, imgatuzumab, sapitinib, TAS-2913, theiiatinib, XGFR-2421, HM-61713B, epitinib, NRC-2694, MLBS-42, JRP-890, cetuximab, AL-6802, TAK-285, BGB-102, AEE788, gefitinib, DMS-3008, TX-2036, KI-6783, KI-6896; or a pharmaceutically acceptable salt, hydrate, solvate or prodrug thereof; or any combination thereof. The PLK1 inhibitor can be onvansertib (NMS-P937), BI2536, volasertib (BI 6727), GSK461364, adavosertib (AZD1775), CYC140, HMN-176, HMN-214, rigosertib (ON-01910), MLN0905, TKM-080301, TAK-960, GTPL10072, Ro3280; or a pharmaceutically acceptable salt, hydrate, solvate or prodrug thereof; or any combination thereof. The first and/or second EGFR inhibitor can be cetuximab or panitumumab and/or the PLK1 inhibitor can be onvansertib. The first and second EGFR inhibitors can be the same. The first and second EGFR inhibitors can be different.

In some embodiments, the instructions comprise instructions for administering PLK-1 inhibitor (e.g., onvansertib) orally. In some embodiments, the instructions comprise instructions for administering EGFR inhibitor (e.g., cetuximab or panitumumab) intravenously or by intraperitoneal route.

In some embodiments, the instructions comprise instructions for subjects who have received a prior EGFR inhibitor treatment. In some embodiments, the instructions comprise instructions for subjects who did not respond to treatment with an EGFR inhibitor alone. In some embodiments, the instructions comprise instructions for subjects who are known to be resistant to an EGFR inhibitor therapy.

In some embodiments, the instructions comprise instructions the subject has received at least one prior treatment for the cancer. In some embodiments, the prior treatment does not comprise the use of a PLK1 inhibitor, or both. In some embodiments, the instructions comprise instructions the subject was in remission for the cancer. In some embodiments, the subject in remission for cancer was in complete remission (CR), or in partial remission (PR).

The instructions can comprise instructions for administering the PLK-1 inhibitor or each of PLK-1 inhibitor and the EGFR inhibitor (e.g., the first EGFR inhibitor) to the subject in a cycle of at least once or twice within a week. In some embodiments, the instructions comprise instructions for administering the PLK-1 inhibitor or each of PLK-1 inhibitor and the EGFR inhibitor (e.g., the first EGFR inhibitor) to the subject in a cycle of at least five times within a week. In some embodiments, the instructions comprise instructions for administering the PLK-1 inhibitor or each of PLK-1 inhibitor and the EGFR inhibitor (e.g., the first EGFR inhibitor) in a cycle of at least 7 days. In some embodiments, each cycle of treatment is at least about 14 days to about 21 days. In some embodiments, each cycle of treatment is from about 21 days to about 28 days. In some embodiments, each cycle of treatment is from about 28 days to about 35 days. In some embodiments, each cycle of treatment is from about 35 days to about 42 days. In some embodiments, each cycle of treatment is from about 42 days to about 49 days. In some embodiments, the instructions comprise instructions for administering the PLK-1 inhibitor on at least four days in the cycle. In some embodiments, the instructions comprise instructions for not administering the PLK-1 inhibitor on at least one day in the cycle. In some embodiments, the instructions comprise instructions for administrating the PLK-1 inhibitor. In some embodiments, the instructions comprise instructions for administrating the EGFR inhibitor (e.g., the first EGFR inhibitor, e.g., cetuximab) once or twice a week. In some embodiments, the instructions comprise instructions for administrating PLK-1 inhibitor or the PLK-1 inhibitor and the EGFR inhibitor for at least two cycles.

In some embodiments, the instructions comprise dosing guidelines for administering the PLK-1 inhibitor and the EGFR inhibitor. In some embodiments, the instructions comprise instructions for administering the PLK1 inhibitor at 8 mg/m²-90 mg/m². In some embodiments, the instructions comprise instructions for administering the PLK1 inhibitor at 5 mg-200 mg. In some embodiments, the instructions comprise instructions for administering the PLK1 inhibitor (e.g., onvansertib) at a dose from about 10 mg/kg of body weight to about 80 mg/kg of body weight, optionally at a dose from about 20 mg/kg of body weight to about 60 mg/kg of body weight, optionally at a dose from about 30 mg/kg of body weight to about 50 mg/kg of body weight. In some embodiments, the first EGFR inhibitor can be administered daily or weekly at a drug/body surface area unit dose of about 15 mg/m² to about 500 mg/m². In some embodiments, the instructions comprise instructions for administration of the PLK-1 inhibitor by oral route.

For example, in some embodiments, the first EGFR inhibitor is an antibody (e.g., cetuximab or panitumumab) and can be administered at, or at about 5 mg/m², 6 mg/m², 7 mg/m², 8 mg/m², 9 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 205 mg/m², 210 mg/m², 215 mg/m², 220 mg/m², 225 mg/m², 230 mg/m², 235 mg/m², 240 mg/m², 245 mg/m², 250 mg/m², 300 mg/m², 350 mg/m², 400 mg/m², 450 mg/m², 500 mg/m², or a number or a range between any two of these values. In some embodiments, the first EGFR inhibitor is an antibody (e.g., cetuximab or panitumumab) and can be administered at, or at about 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 300 mg, 350 mg, 400 mg, 450 mg, 500 mg, or a number or a range between any two of these values. In some embodiments, the instructions comprise instruction for administration of the EGFR inhibitor (e.g., the first EGFR inhibitor, e.g., cetuximab) by intravenous or intraperitoneal route.

### EXAMPLES

Some aspects of the embodiments discussed above are disclosed in further detail in the following examples, which are not in any way intended to limit the scope of the present disclosure.

### Example 1

### Methods for Treating Colorectal Cancer

As provided herein, the PLK1 inhibitor, Onvansertib, is active as monotherapy and in combination with Cetuximab in RAS Wild-type Colorectal Cancer Patient-derived Xenografts.

Cetuximab and panitumumab are monoclonal antibodies targeting the epidermal growth factor receptor (EGFR), that provide clinical benefit to metastatic colorectal cancer (mCRC) patients with RAS wild-type (RAS^{WT}) tumors. Unfortunately, intrinsic or acquired resistance to these therapies limits their clinical effectiveness, necessitating the development of more effective therapeutic strategies. Onvansertib is an oral, small molecule, selective inhibitor of the PLK1 kinase, currently in clinical development for KRAS-mutant mCRC. The aim of this study was to assess the anti-tumor activity of onvansertib monotherapy and in combination with cetuximab in RAS^{WT} CRC patient-derived xenograft (PDX) models.

Twenty RAS^{WT} CRC PDXs were selected and engrafted in nude mice. Once tumors reached 200-350 mm³, mice were treated with vehicle, onvansertib (60 mg/kg, QD), cetuximab (20 mg/kg, BIW) or the combination for 18 days. PDX models were chosen based on their sensitivity to cetuximab, resulting in a selection of 7 cetuximab-sensitive (CetuxS) and 13 cetuximab-resistant (CetuxR) PDXs, including 7 with intrinsic resistance and 6 with acquired-resistance. Tumor volume change from baseline (TVC) was calculated as 100%×(Vt−Vo)/Vo and tumor growth inhibition (TGI) as 100%×(TVCcontrol−TVCtreated)/TVCcontrol. Tumor regression was defined as TVCD18<0 and tumor stasis 0≤TVCD18<100.

Cetuximab sensitivity was confirmed in the selected models, as cetuximab induced tumor regression in all the CetuxS PDXs, while no or limited activity was observed in the resistant models (median TGI=29%, IQR 16-61). Onvansertib exhibited potent anti-tumor activity in 17 out of 20 (85%) PDXs, resulting in tumor regression (n=11) or tumor stasis (n=6). Onvansertib TGI on Day 18 was not significantly different in CetuxS PDXs (median TGI 102, IQR 76-103) compared to CetuxR PDXs (median TGI 108, IQR 74-124), supporting that onvansertib anti-tumor activity is independent of the sensitivity/resistance to cetuximab. The combination of onvansertib and cetuximab induced tumor regression in 18 (90%) PDXs. The combination showed significantly improved efficacy compared to individual therapies in some of the models.

Onvansertib monotherapy displayed potent anti-tumor activity in RAS^{WT} CRC PDX models, independently of their sensitivity to cetuximab. Additionally, onvansertib combined with cetuximab exhibited either comparable or superior anti-tumor activity than the monotherapies. Collectively, this data supports the clinical development of the PLK1 inhibitor onvansertib for RAS^{WT} mCRC.

### Example 2

### Onvansertib is Active as Monotherapy and in Combination with EGFR Inhibitors for Treatment of Colorectal Cancer

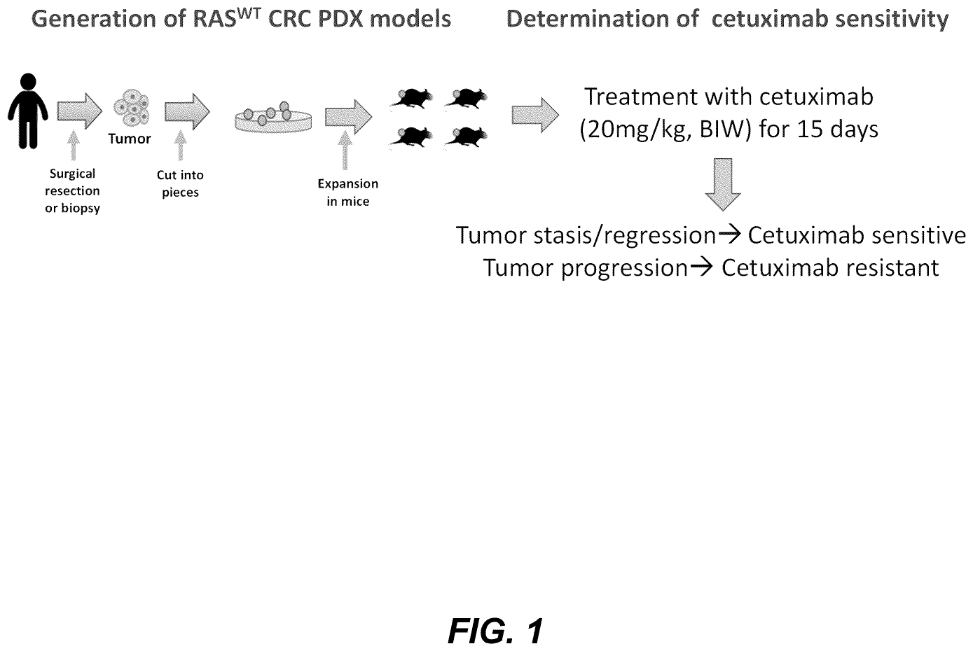

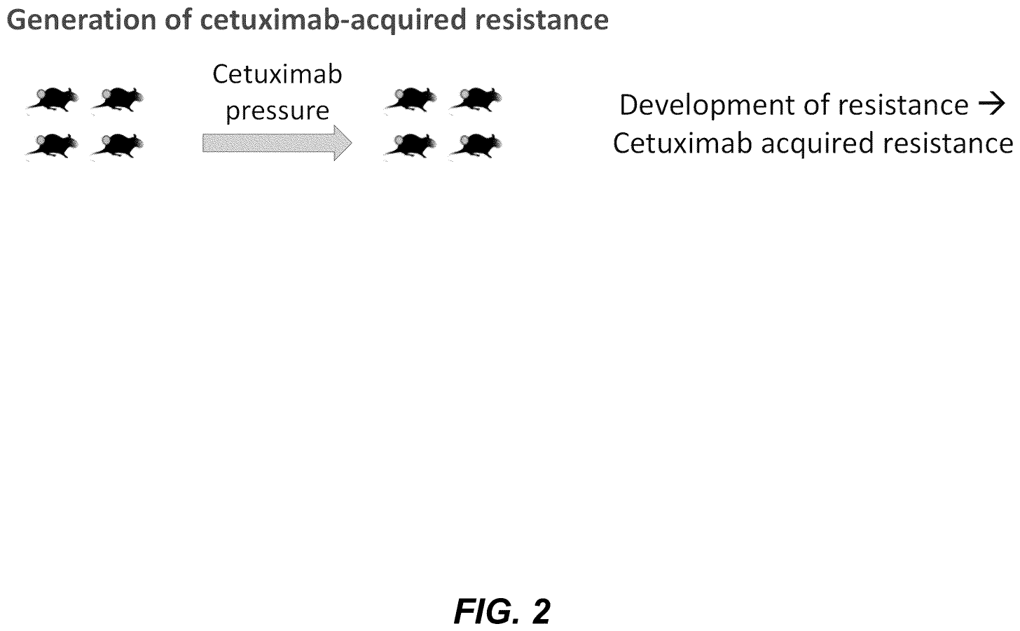

Provided in this example are methods for use of PLK1 inhibitor as monotherapy or combination with EGFR inhibitor for treatment of cancer. For assessment of onvansertib (Onv) and cetuximab (cetux) antitumor activity the following RAS^{WT} colorectal cancer patient derived xenograft (PDX) models were selected: seven PDXs sensitive to cetux, seven PDXs with intrinsic resistance to cetux, and six PDXs with acquired resistance to cetux (See, FIG. 1-FIG. 2).

Treatment (n=5-7 mice/arm) was for 18-19 days, and included the following groups: Vehicle, Cetuximab (intraperitoneal, IP, 20 mg/kg, twice per week), Onvansertib (oral, 60 mg/kg, daily), and Onv plus Cetux.

### PDXs Sensitive to Cetuximab

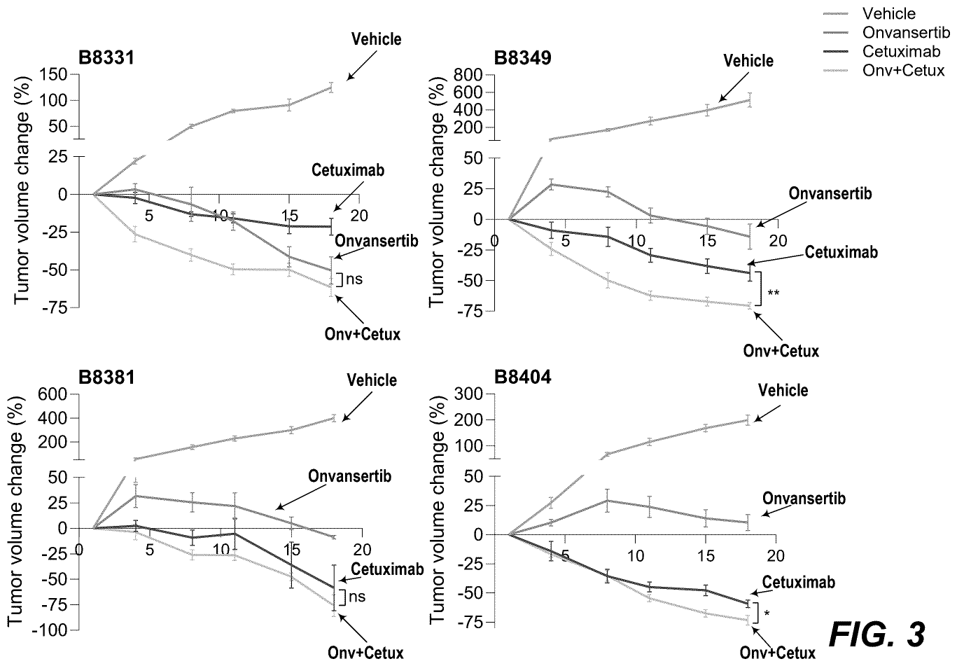

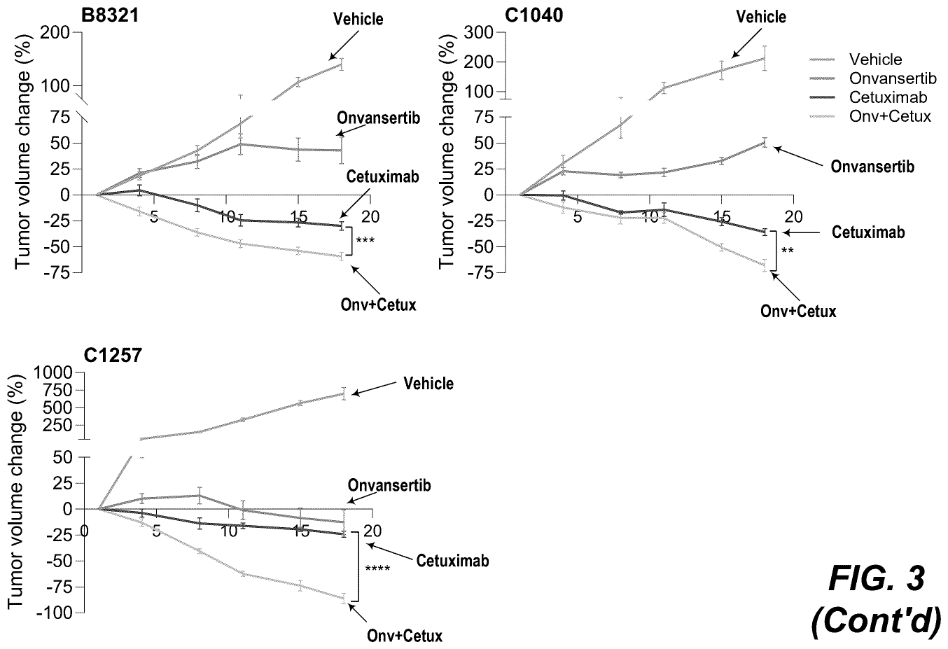

As expected, all models responded to cetuximab. Onvansertib induced tumor stasis or regression in five models and tumor growth inhibition in two models. Combination treatment resulted in tumor regression in the seven models. Antitumor activity of the combination was slightly increased compared to cetuximab single agent in 5/7 models (See, FIG. 3).

PDXs with Intrinsic Resistance to Cetuximab

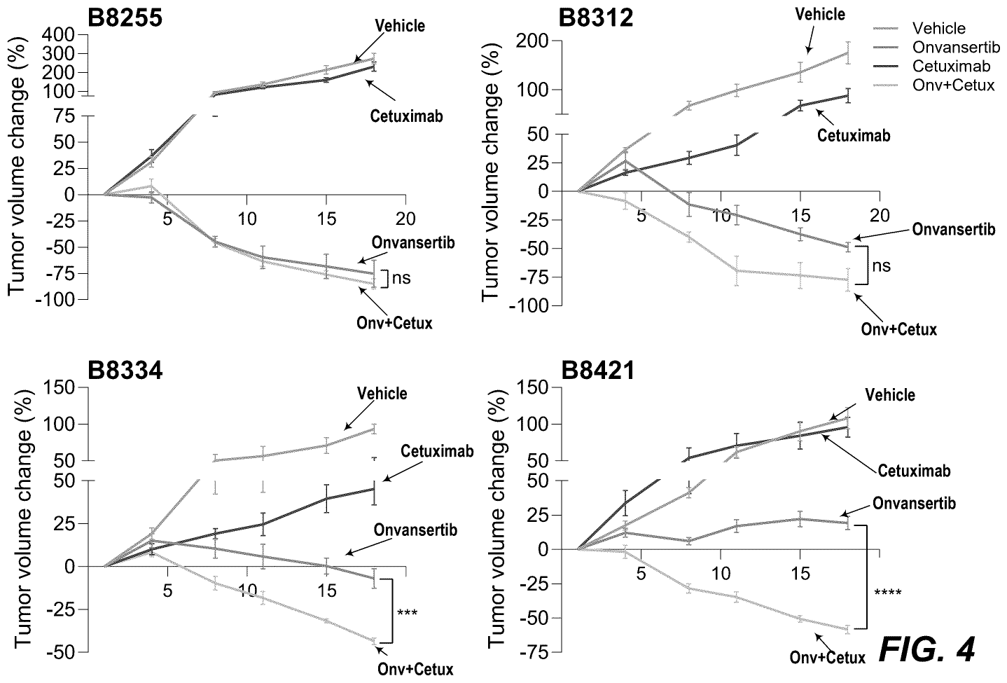

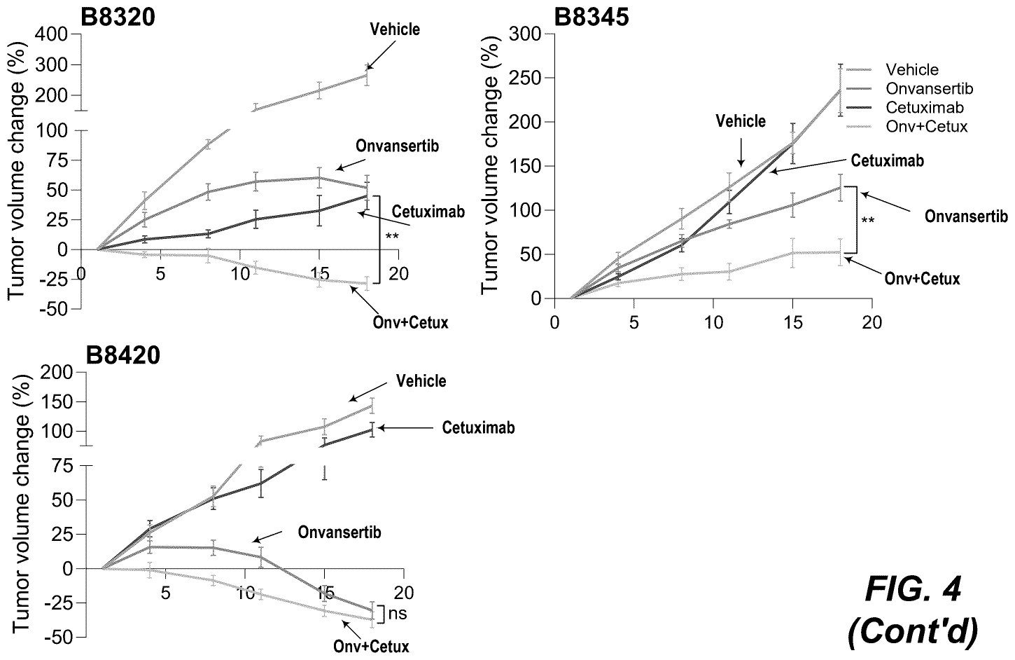

Cetuximab resistance was confirmed in six of the seven models. Onvansertib induced tumor stasis or regression in 5/7 models and tumor growth inhibition in two models. Combination treatment resulted in tumor regression in six models and tumor growth inhibition in one model. Antitumor activity of the combination was significantly greater compared to monotherapies in 4/7 models (See, FIG. 4).

PDXs with Acquired Resistance to Cetuximab

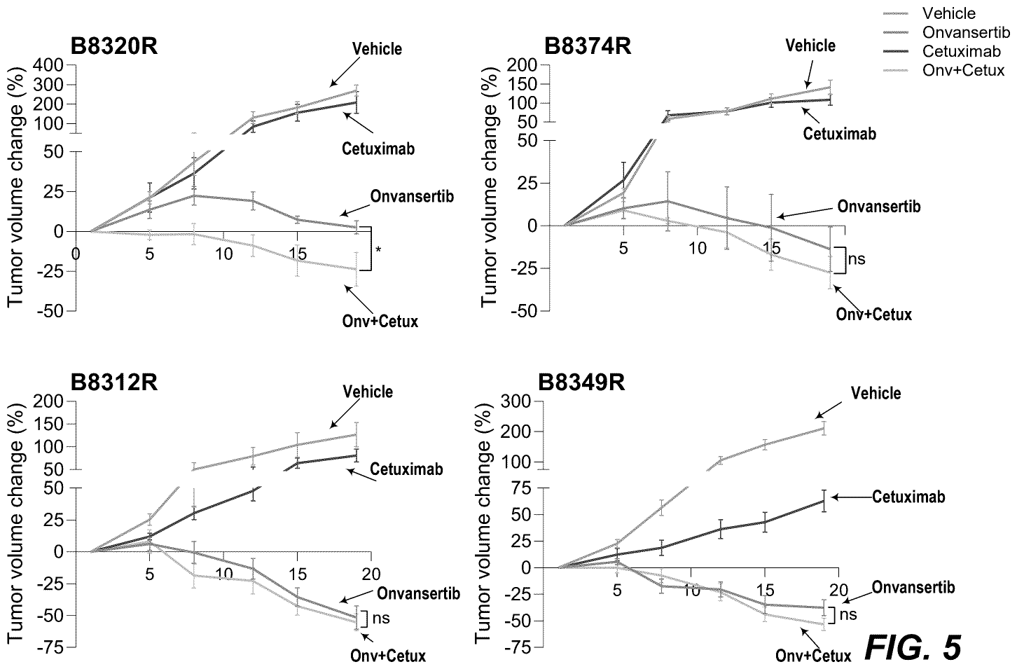

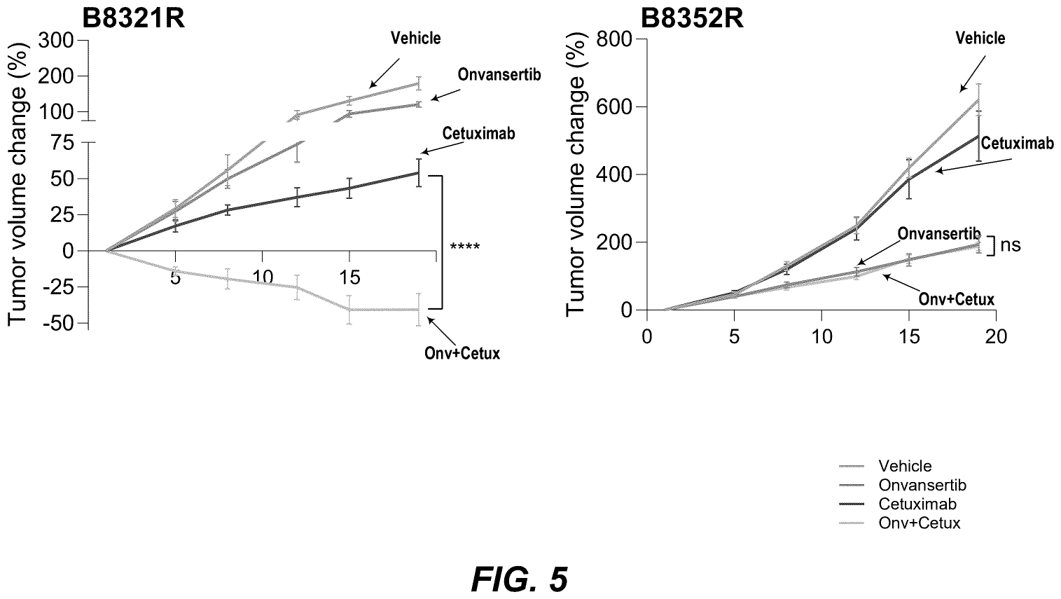

Four models were resistant to cetuximab and two showed partial response. Onvansertib induced tumor stasis or regression in 4/6 models and tumor growth inhibition in one model. The combination treatment resulted in tumor stasis or regression in 5/6 models, and superior antitumor activity compared to single agents in two models (See, FIG. 5).

Onvansertib Monotherapy and in Combination with Cetuximab across Models

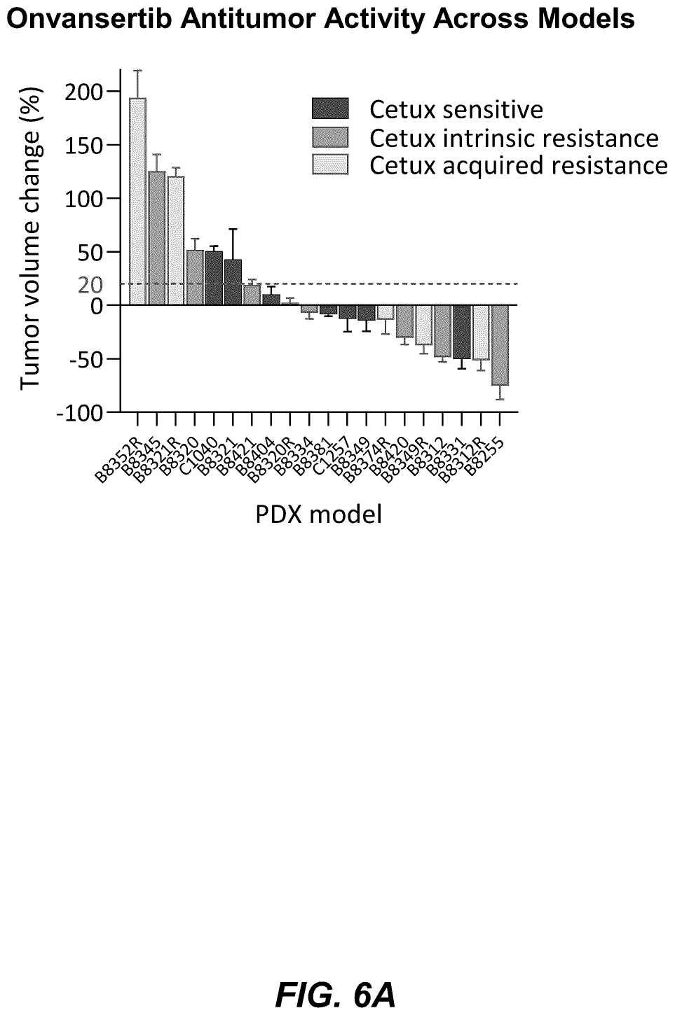

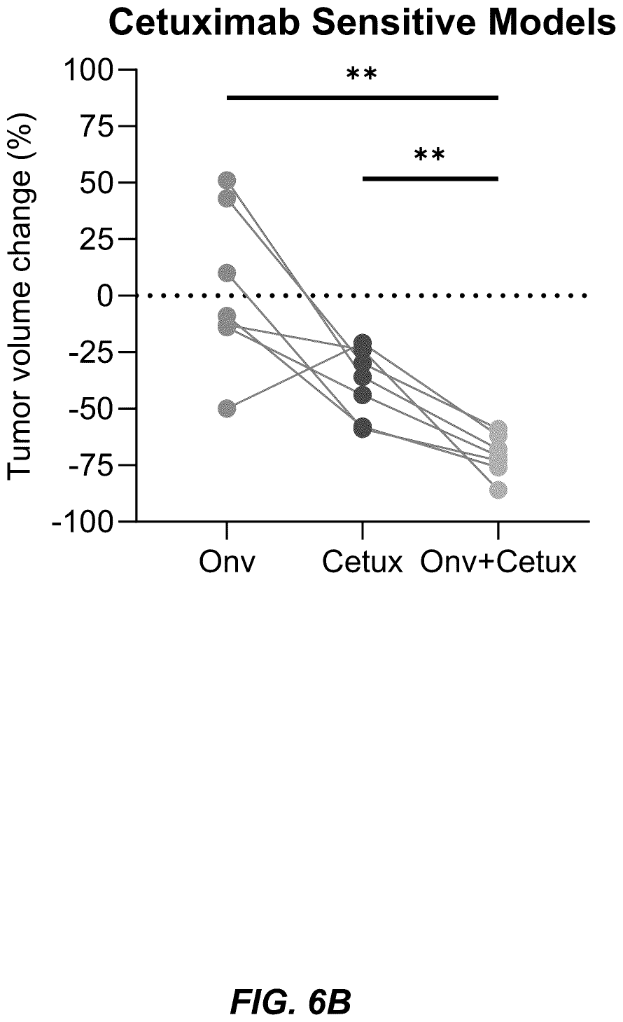

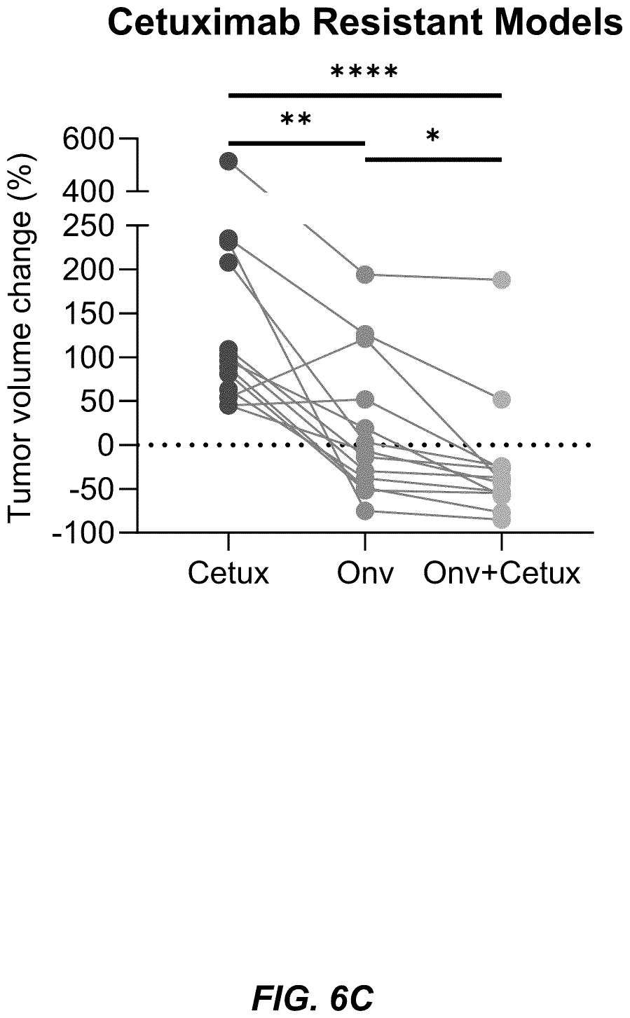

Onvansertib induced tumor regression (n=11) or stasis (n=3) in 14 (70%) of the 20 models. Onvansertib potent antitumor activity was observed in PDX models sensitive to cetuximab (5/7, 71%) and resistant to cetuximab (9/13, 69%). Onvansertib in combination with cetuximab induced tumor regression in 18 (90%) of the 20 models. Overall, the antitumor activity of the combination was superior compared to monotherapies in both cetuximab sensitive and resistant models (See, FIG. 6A-FIG. 6C).

Onvansertib displayed robust antitumor activity in RAS^{WT} CRC PDXs and induced tumor stasis or regression in 70% (14/20) of the models. Efficacy was independent of cetuximab sensitivity, similar antitumor activity observed in cetuximab sensitive and resistant models. The onvansertib and cetuximab combination was highly effective and induced tumor stasis or regression in 90% (18/20) of the models and also resulted in enhanced efficacy compared to monotherapies.

Genomic and proteomic analyses are ongoing to identify potential biomarkers of response and resistance to onvansertib. Collectively, these data support the clinical development of onvansertib as a potential treatment for RAS wild-type colorectal cancer.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of treating RAS wild type (RAS^{WT}) colorectal cancer, the method comprising:
administering onvansertib in combination with a first EGFR inhibitor to a subject with a RAS^{WT} colorectal cancer, thereby inhibiting or reducing progression of the RAS^{WT} colorectal cancer in the subject, wherein and the subject is a human.

2. The method of claim 1, wherein the subject with the RAS^{WT} colorectal cancer is resistant to treatment with the EGFR inhibitor or has stable or progressive disease following treatment with the EGFR inhibitor.

3. The method of claim 1, wherein the subject with the RAS^{WT} colorectal cancer is resistant to or does not respond effectively to a second EGFR inhibitor.

4. The method of claim 3, wherein the resistance is acquired resistance or intrinsic resistance.

5. The method of claim 3, wherein the subject with the RAS^{WT} colorectal cancer is known to be resistant to the second EGFR inhibitor.

6. The method of claim 1, wherein onvansertib and the first EGFR inhibitor are co-administered simultaneously or sequentially.

7. The method of claim 1, wherein onvansertib, the first EGFR inhibitor, or both are administered to the subject in a cycle of 7 days, 14 days, 28 days, 35 days, 42 days, or 49 days.

8. The method of claim 1, wherein onvansertib is administered daily, and the first EGFR inhibitor is administered twice a week.

9. The method of claim 1, wherein the onvansertib is administered on at least five days, at least ten days, or at least fifteen days in a cycle and is not administered on at least one day, at least three days, or at least seven days in a cycle.

10. The method of claim 3, wherein the first and/or the second EGFR inhibitor is erlotinib, lapatinib, AZD8931, WZ4002, panitumumab, vandetanib, icotinib, afatinib, brigatinib, CO-1688, AZD-4769, poziotinib, CUDC-101, S-222611, AC-480, imgatuzumab, sapitinib, TAS-2913, theiiatinib, XGFR-2421, HM-61713B, epitinib, NRC-2694, MLBS-42, JRP-890, cetuximab, AL-6802, TAK-285, BGB-102, AEE788, gefitinib, DMS-3008, TX-2036, KI-6783, KI-6896; or a pharmaceutically acceptable salt, hydrate, solvate or prodrug thereof; or any combination thereof.

11. The method of claim 3, wherein the first and/or the second EGFR inhibitor is cetuximab or panitumumab.

12. The method of claim 3, wherein the first and the second EGFR inhibitors are the same or different.

13. The method of claim 1, further comprising determining responsiveness of the subject to the treatment with onvansertib and the first EGFR inhibitor.

14. The method of claim 1, wherein the volume of one or more tumors in the subject increases by at most 25% relative to the volume of the one or more corresponding tumors prior to the administering, following at least one cycle of treatment.

15. The method of claim 1, wherein the volume of one or more tumors in the subject decreases by at least 25% relative to the volume of the one or more corresponding tumors prior to the administering, following at least one cycle of treatment.

16. The method of claim 1, wherein the subject with the RAS^{WT} colorectal cancer is resistant to the first EGFR inhibitor.

17. The method of claim 1, wherein the first EGFR inhibitor is cetuximab.

18. The method of claim 19, wherein the subject with the RAS^{WT} colorectal cancer is resistant to cetuximab.

19. A method of sensitizing cancer cells to an EGFR inhibitor, the method comprising: contacting cancer cells with a composition comprising onvansertib, thereby sensitizing the cancer cells to the EGFR inhibitor, wherein the cancer cells are RAS^{WT} colorectal cancer cells or cells of EGFR-amplified cancer, and wherein the EGFR-amplified cancer is a cancer with higher EGFR mRNA and/or protein levels as compared to a healthy tissue.

20. The method of claim 19, comprising determining sensitization of the cancer cells to the EGFR inhibitor after being contacted with the composition.
